(19) United States
(12) Patent Application Publication
(10) Pub. No.: US 2013/0237582 A1
(43) Pub. Date: Sep. 12, 2013

(54) PHARMACEUTICAL COMBINATION OF PACLITAXEL AND A CDK INHIBITOR

(71) Applicants: Maggie Joyce Rathos, Mumbai (IN); Kalpana Sanjay Joshi, Thane (IN)

(72) Inventors: Maggie Joyce Rathos, Mumbai (IN); Kalpana Sanjay Joshi, Thane (IN)

(73) Assignee: Piramal Enterprises Limited, Mumbai (IN)

(21) Appl. No.: 13/988,240

(22) PCT Filed: Nov. 18, 2011
(86) PCT No.: PCT/IB11/55179

§ 371 Date: May 17, 2013

Related U.S. Application Data
(60) Provisional application No. 61/415,507, filed on Nov. 19, 2010.

(51) Int. Cl. A61K 31/4025; A61K 31/337

(52) U.S. Cl. CPC A61K 31/4025; A61K 31/337
USPC 514/422

(57) ABSTRACT

The present invention relates to a pharmaceutical combination comprising paclitaxel, or its pharmaceutically acceptable salt; and at least one cyclin dependent kinase (CDK) inhibitor represented by a compound of formula I (as described herein) or a pharmaceutically acceptable salt thereof, for use in the treatment of triple negative breast cancer (TNBC). The present invention relates to a method for the treatment of breast cancer, particularly triple negative breast cancer, by administration to a patient in need thereof, a therapeutically effective amount of a pharmaceutical combination comprising a cytotoxic antineoplastic agent, paclitaxel, and at least one cyclin dependent kinase (CDK) inhibitor; wherein said combination on administration exhibits synergistic effects.

### FIELD OF THE INVENTION

The present invention relates to a pharmaceutical combination comprising paclitaxel, or its pharmaceutically acceptable salt; and at least one cyclin dependent kinase (CDK) inhibitor represented by a compound of formula I (as described herein) or a pharmaceutically acceptable salt thereof, for use in the treatment of triple negative breast cancer (TNBC). The invention also relates to a method of treating triple negative breast cancer in a subject comprising administering to the subject a pharmaceutical combination comprising a therapeutically effective amount of paclitaxel, or its pharmaceutically acceptable salt; and a therapeutically effective amount of at least one cyclin dependent kinase (CDK) inhibitor represented by a compound of formula I (as described herein) or a pharmaceutically acceptable salt thereof.

### BACKGROUND OF THE INVENTION

Cancer is a general term used to describe diseases in which abnormal cells divide without control. Cancer cells can invade adjacent tissues and can spread through the bloodstream and lymphatic system to other parts of the body. There are different types of cancers such as the bladder cancer, breast cancer, colon cancer, rectal cancer, head and neck cancer, endometrial cancer, kidney (renal cell) cancer, leukemia, small cell lung cancer, non-small cell lung cancer, pancreatic cancer, prostate cancer, thyroid cancer, skin cancer, Non-Hodgkin's Lymphoma and melanoma. Currently there are many treatments available for cancer than ever before, including chemotherapy, radiation, surgery, hormonal therapy, immune therapy and gene therapy. Chemotherapy is the most routinely used treatment for cancer.

The most widely used chemotherapeutic agents (the antineoplastic agents) include paclitaxel, docetaxel, doxorubicin, etoposide, carboplatin, cisplatin, topotecan and gemcitabine. These antineoplastic agents have been successfully used for the treatment of different cancers. However, in due course of time, some cancer patients have been found to develop resistance to monotherapy involving use of such standard antineoplastic agents. Tolerance or resistance to a drug represents a major impediment to successful treatment. Such resistance is often considered as either intrinsic (i.e. present at the onset of treatment) or acquired (i.e. occurs during the course of chemotherapy). A study involving exposure of human non-small cell lung cancer cells (NCI-H460) to gradually increasing concentrations of doxorubicin reported appearance of a new cell line (NCI-H460/R) that was resistant to doxorubicin and cross-resistant to etoposide, paclitaxel, vinblastine and epirubicin (J. Chemother., 2006, 18, 1, 66-73). Gemcitabine was considered to be the most clinically active drug for the treatment of pancreatic cancer, however it failed to significantly improve the condition of pancreatic cancer patients because of the pre-existing or acquired chemo resistance of the tumor cells to the drug (Oncogene, 2003, 22, 21, 3243-51).

Another problem observed or prevalent in cancer treatments is the severe toxicity associated with most of the antineoplastic agents. Despite the incidence of resistance and severe toxicity associated with the conventional antineoplastic agents e.g. gemcitabine and paclitaxel, these agents still continue to be important in the cancer treatment because they have the ability to reduce tumor mass. In order to improve the response rate and prevent toxicity associated with the conventional antineoplastic agents, new therapeutic approaches are being evaluated.

One such approach is directed to a protocol involving combination of different anticancer agents. An optimal combination chemotherapy protocol may result in increased therapeutic efficacy, decreased host toxicity, and minimal or delayed drug resistance. When drugs with different toxicities are combined, each drug can be used at its optimal dose, helping to minimise intolerable side effects. Some of the antineoplastic agents have been found to be synergistically effective when used in combination with other anticancer agents than when used as a monotherapy.

Cyclophosphamide and 5-fluorouracil act synergistically in ovarian clear cell adenocarcinoma cells (Cancer Lett., 2001, 162, 1, 39-48). Combination chemotherapy can also be advantageously used for treating cancers in advanced stages which are difficult to treat with monotherapy, radiation or surgical treatment, for example, a combination of paclitaxel and gemcitabine has been reported for the treatment of metastatic non-small cell lung cancer (Cancer, 2006, 107, 5, 1050-1054). Gemcitabine and carboplatin combination chemotherapy was relatively safe and effective for treating elderly patients with non-small cell lung cancer (Cancer Res. Treat., 2008, 40, 116-120). Gemcitabine plus carboplatin combination is active in advanced TCC (transitional cell carcinoma) with acceptable toxicity (BMC Cancer, 2007, 7, 98). Treatment with gemcitabine and carboplatin significantly improves the progression-free survival of patients with platinum-sensitive recurrent ovarian cancer (Int. J. Gynecol. Cancer, 2005, 15 (Suppl. 1), 36-41).

Recently, combination of one or more standard antineoplastic agents such as paclitaxel, cisplatin etc. with a molecularly targeted anticancer agent for the treatment of cancer has been tried out to improve drug response rates and to address resistance to the antineoplastic agents. Molecularly targeted agents e.g. imatinib mesylate, flavopiridol etc. modulate proteins such as kinases whose activities are more specifically associated with cancerous cells. Researches over a long period of time have proven that the members of the cyclin-dependent kinase (CDK) family play key roles in various cellular processes. There are 11 members of the CDK family known till now. Among these, CDK1, CDK2, CDK3, CDK4 and CDK6 are known to play important roles in the cell cycle (Adv. Cancer Res., 1995, 66, 181-212). CDKs are activated by forming noncovalent complexes with cyclins such as A-type, B-type, C-type, D-type (D1, D2, and D3), and E-type cyclins. Each isozyme of this family is responsible for particular aspects (cell signaling, transcription, etc.) of the cell cycle, and some of the CDK isozymes are specific to certain kinds of tissues. Aberrant expression and overexpression of these kinases are evidenced in many disease conditions. A number of compounds having potentially useful CDK inhibitory properties have been developed and reported in the literature.

Flavopiridol is the first potent inhibitor of cyclin-dependent kinases (CDKs) to reach clinical trial. Flavopiridol has been found to potentiate synergistically the cytotoxic response of the conventional cytotoxic antineoplastic agents in a variety of cancer cell-lines. For example, combined docetaxel and flavopiridol treatment for lung cancer cells has been reported in Radiother. Oncol., 2004, 71, 2, 213-21 and for the treatment of gastric cancer in Mol. Cancer. Ther., 2003, 2, 6, 549-55. PCT publication WO2008139271 discloses the combinations of a CDK inhibitor, (+)-trans-2-(2-Chlorophenyl)-5,7-dihydroxy-8-(2-hydroxymethyl-1-methyl-pyrrolidin-3-yl)-chromen-4-one hydrochloride with cytotoxic neoplastic agents such as doxorubicin, docetaxel, paclitaxel and gemcitabine for the treatment of non-small cell lung carcinoma and pancreatic cancer.

Although various treatment options are available for the treatment of cancers, this disease still remains one of the most fatal diseases. Although, all the types of cancers are fatal, breast cancer still remains a type of fatal cancer. In fact, in women, breast cancer is among the most common cancers and is the fifth most common cause of cancer deaths. Different forms of breast cancers can have remarkably different biological characteristics and clinical behavior. Thus, classification of a patient's breast cancer has become a critical component for determining a treatment regimen. Breast cancer patients fall into three main groups:
(i) those with hormone receptor-positive tumors who are managed with a number of estrogen receptor (ER)-targeted therapy options ±chemotherapy;
(ii) those with HER2+ tumors, who will, in addition, receive HER2-directed therapy with trastuzumab or, in some situations, lapatinib; and
(iii) those with hormone receptor [ER and progesterone receptor (PR)]-negative and HER2) breast cancers, for whom chemotherapy is the only modality of systemic therapy available.

Currently, trastuzumab has been developed as a targeted therapy for breast cancer patients. Studies have shown that the expression profiles of breast cancer display a systematic variation and allow classification of breast cancer into five main groups, two of them ER+(luminal A and B) and three ER− groups [normal breast-like, ERBB2 (also known as HER2) and ‘basal-like’]. It has been shown that the basal-like group is enriched for tumors that lack expression of hormone receptors and of HER2 and has a more aggressive clinical behavior, a distinctive metastatic pattern and a poor prognosis despite responding to conventional neoadjuvant and adjuvant chemotherapy regimens. Based on the above it is clear that the interest in triple-negative breast cancers stems from (i) the lack of tailored therapies for this group of breast cancer patients and (ii) overlap with the profiles of basal-like cancers (Histopathology, 2008, 52, 108-118).

Triple-negative breast cancer (TNBC) i.e. tumors that are estrogen receptor (ER)-negative and progesterone receptor (PR)-negative and do not overexpress human epidermal growth factor receptor 2 (HER2) account for approximately 15% of breast cancers, with approximately 170,000 cases reported worldwide in 2008. Triple-negative breast cancers are significantly more aggressive (metastatic) than tumors pertaining to other molecular subgroups. TNBC does not express estrogen (ER), progesterone (PR) and HER2 receptors, therefore, they are resistant to currently available targeted treatment, including hormonal and HER2-targeted therapies. Patients with basal-like or triple negative cancers have a significantly shorter survival following the first metastatic event when compared with those with non-basal-like/no-triple negative patients. A vast majority of tumors arising in BRCA1 germ-line mutation carriers have morphological features similar to those described in basal-like cancers and they display a triple negative and basal like phenotype.

TNBC constitutes one of the most challenging groups of breast cancers. The only systemic therapy currently available for patients with such cancers is chemotherapy. However, the survival of patients with such tumors is still poor and their management may, therefore, require a more aggressive intervention. As a result the development of targeted therapies for TNBC is of considerable importance. Recent trials have shown that poly (ADP-ribosyl)ation polymerase (PARP) inhibitor, BSI-201 (currently known as Iniparib developed by Sanofi-Aventis) is highly effective in TNBC (Maturitas, 2009, 63, 269-274). Also TNBC is characterized by elevated levels of PARP. These characteristics have suggested that PARP inhibition might be able to potentiate the effects of chemotherapy-induced DNA damage in TNBC (Community Oncology, 2010, 7, 5, 2, 7-10; Clinical Advances in Hematology and Oncology, 7, 7, 441-443).

Although triple-negative breast cancers are reported to respond to chemotherapy, survival of patients with such tumors is still poor and their management may therefore require a more aggressive alternative intervention. Thus, the development of biologically informed systemic therapies and targeted therapies for triple-negative breast cancers is of paramount importance and may prove to be achievable by understanding the complexity of this heterogeneous group of tumors and using combination therapy (Histopathology, 2008, 52, 108-118).

In view of the above discussion and considering that treatment options for treating triple negative breast cancer are very limited, a need remains for additional treatment options and methods for treating TNBC.

### SUMMARY OF THE INVENTION

In one aspect, the present invention relates to a pharmaceutical combination comprising a therapeutically effective amount of paclitaxel, or its pharmaceutically acceptable salt; and a therapeutically effective amount of a cyclin dependent kinase (CDK) inhibitor represented by a compound of formula I (as described herein) or a pharmaceutically acceptable salt thereof, for use in the treatment of triple negative breast cancer (TNBC).

In one aspect, the present invention relates to a method of treating triple negative breast cancer in a subject comprising administering to the subject a therapeutically effective amount of paclitaxel, or a pharmaceutically acceptable salt thereof; in combination with a therapeutically effective amount of a cyclin dependent kinase (CDK) inhibitor represented by a compound of formula I (as described herein) or a pharmaceutically acceptable salt thereof.

In another aspect, the present invention relates to a method of treating triple negative breast cancer in a subject comprising administering to the subject a therapeutically effective amount of paclitaxel, or its pharmaceutically acceptable salt; followed by a therapeutically effective amount of the CDK inhibitor represented by a compound of formula I or a pharmaceutically acceptable salt thereof, to the subject.

In a further aspect, the present invention relates to use of a pharmaceutical combination comprising a therapeutically effective amount of paclitaxel or its pharmaceutically acceptable salt and a therapeutically effective amount of a CDK inhibitor represented by the compound of formula I or a pharmaceutically acceptable salt thereof for the treatment of triple negative breast cancer.

In yet another further aspect, the present invention relates to use of a pharmaceutical combination comprising paclitaxel or its pharmaceutically acceptable salt and a CDK inhibitor represented by the compound of formula I or a pharmaceutically acceptable salt thereof; for the manufacture of a medicament for treating triple negative breast cancer

Other aspects and further scope of applicability of the present invention will become apparent from the detailed description to follow.

### BRIEF DESCRIPTION OF THE DRAWINGS

FIG. 1: Effect of Compound A on colony formation in breast cancer cell lines (MDA-MB-231, MDA-MB-468 and MCF-7)

FIG. 2: Effect of Compound A on MCTS formation in MCF-7 breast cancer cell line

FIG. 3A: Time dependent effect of Compound A on cell cycle progression and apoptosis in MCF-7 (Her2-, BRCA+/−allelic loss) cell line

FIG. 3B: Time dependent effect of Compound A on cell cycle progression and apoptosis in MDA-MB-231 cell line

FIG. 4: Expression of antiapoptotic protein Bcl-2 in MCF-7 and MDA-MB-231 cell lines treated with Compound A

FIG. 5A: Effect of Compound A on MDA-MB-231 cell line (different phases of the cell cycle)

FIG. 5B: Effect of Compound A on MDA-MB-468 cell line

FIG. 5C: Effect of BSI-201 on TNBC MDA-MB-231 and MDA-MB-468 cell lines

FIG. 6A: Cyclin D1 level in various breast cancer cell lines

FIG. 6B: Effect of Compound A on MCF-7 cell cycle proteins and CDK4 kinase activity

FIG. 7: Effect of Compound A on PARP enzyme activity in breast cancer cell lines (MDA-MB-231 and MDA-MB-468) as measured by PAR polymers

FIG. 8: Effect of Compound A (24 h) on PARP and cell cycle proteins in two TNBC cell lines (MDA-MB-231 and MDA-MB-468)

FIG. 9: Effect of Compound A on HIF-1α inhibition in the U251 HRE and U251 pGL3 cell lines

FIG. 10: Effect of Compound A on VEGF inhibition using the VEGF reporter gene based assay

FIG. 11A: Effect of Compound A on the migration of BT-549 breast cancer cell line

FIG. 11B: Effect of Compound A on the migration of MDA-MB-231 breast cancer cell line

FIG. 11C: Effect of Compound A on the migration of MCF-7 breast cancer cell line

FIG. 12: Effect of Compound A on endothelial tube formation as observed in Endothelial Cell Tube Formation Assay

FIG. 13: Effect of the combination of Paclitaxel for 24 h followed by complete medium (CM)—Group IA/Compound A (IC₅₀)—Group IVA/Sunitinib (IC₅₀)—Group VA for 72 h in MDA-MB-231 cell line

FIG. 14: Effect of the combination of Paclitaxel for 24 h followed by complete medium (CM)—Group IB/Compound A (IC₅₀)—Group WB/Sunitinib (IC₅₀)—Group VB for 72 h in BT-549 cell line

FIG. 15: Effect of the combination of Paclitaxel for 24 h followed by Complete medium (CM)—Group IC/Compound A (IC₅₀)—Group IVC/Sunitinib (IC₅₀)—Group VC for 72 h in MDA-MB-468 cell line

### DETAILED DESCRIPTION OF THE INVENTION

It has now been found that the pharmaceutical combination of the present invention, which comprises paclitaxel, or its pharmaceutically acceptable salt and a CDK inhibitor selected from the compound of formula I (as described herein) or a pharmaceutically acceptable salt thereof; exhibits synergistic effect when used in the treatment of triple negative breast cancer.

In particular, the present invention provides a method of treating, or managing triple negative breast cancer in a subject comprising administering to the subject a therapeutically effective amount of paclitaxel in combination with a therapeutically effective amount of a CDK inhibitor selected from the compounds of formula I.

The CDK inhibitor comprised in the pharmaceutical combination of the present invention is selected from the compound of formula I as described herein. The CDK inhibitors represented by the following formula I are disclosed in PCT Patent Publication No. WO2004004632 (corresponding to U.S. Pat. No. 7,272,193) and PCT Patent Publication No. WO2007148158, which are incorporated herein by reference. The compounds of formula I are CDK inhibitors, which inhibit proliferation of different cancer cells. The compounds of formula I comprised in the pharmaceutical combination of the present invention are effective against various solid and hematological malignancies. The inventors of the present invention observed that combining the compounds of formula I with paclitaxel resulted in an increase in apoptosis, or programmed cell death.

The CDK inhibitors used in the present invention are selected from the compounds represented by the following formula I,

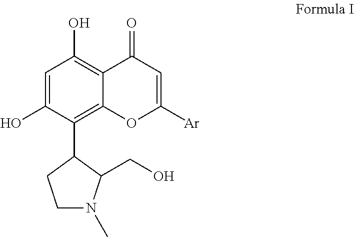

wherein Ar is a phenyl group, which is unsubstituted or substituted by 1, 2, or 3 identical or different substituents selected from: halogen selected from chloro, bromo, fluoro or iodo; nitro, cyano, C₁-C₄-alkyl, trifluoromethyl, hydroxy, C₁-C₄-alkoxy, carboxy, C₁-C₄-alkoxycarbonyl, CONH₂ or NR₁R₂;
wherein R₁ and R₂ are each independently selected from hydrogen or C₁-C₄-alkyl.

Compounds of Formula (I) may be prepared according to the methods disclosed in PCT Publication No. WO2004004632 and PCT Publication No. WO2007148158 which are incorporated herein by reference.

The general process for the preparation of the compounds of Formula (I), or a pharmaceutically acceptable salt thereof, comprises the following steps:

(a) treating the resolved enantiomerically pure (−)-trans enantiomer of the intermediate compound of Formula VIA,

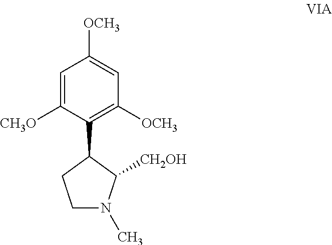

with acetic anhydride in the presence of a Lewis acid catalyst to obtain a resolved acetylated compound of Formula VIIA,

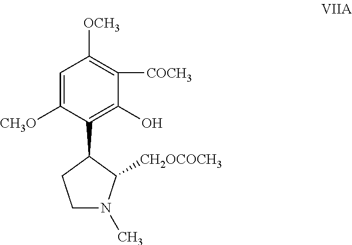

(b) reacting the resolved acetylated compound of Formula VIIA with an acid of Formula ArCOOH or an acid chloride of Formula ArCOCl or an acid anhydride of Formula (ArCO)₂O or an ester of Formula ArCOOCH₃, wherein Ar is as defined hereinabove in reference to the compound of Formula (I), in the presence of a base and a solvent to obtain a resolved compound of Formula VIIIA;

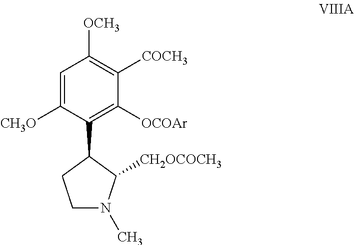

(c) treating the resolved compound of Formula VIIIA with a base in a suitable solvent to obtain the corresponding resolved β-diketone compound of Formula IXA;

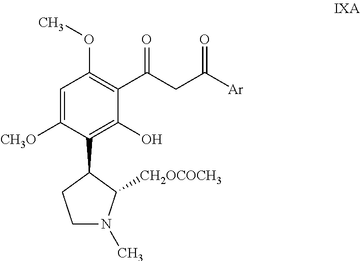

wherein Ar is as defined above;
(d) treating the resolved β-diketone compound of Formula IXA with an acid such as hydrochloric acid to obtain the corresponding cyclized compound of Formula XA,

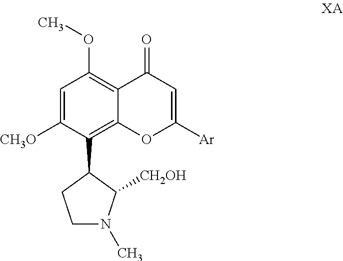

(e) subjecting the compound of Formula XA to dealkylation by heating it with a dealkylating agent at a temperature ranging from 120-180° C. to obtain the (+)-trans enantiomer of the compound of Formula (I) and, optionally, converting the subject compound into its pharmaceutically acceptable salt.

The Lewis acid catalyst utilized in the step (a) above may be selected from: BF₃, Et₂O, zinc chloride, aluminium chloride and titanium chloride.

The base utilized in the process step (b) may be selected from triethylamine, pyridine and a DCC-DMAP combination (combination of N,N′-dicyclohexyl carbodiimide and 4-dimethylaminopyridine).

It will be apparent to those skilled in the art that the rearrangement of the compound of Formula VIIIA to the corresponding β-diketone compound of Formula IXA is known as a Baker-Venkataraman rearrangement (J. Chem. Soc., 1933, 1381 and Curr. Sci., 1933, 4, 214).

The base used in the process step (c) may be selected from: lithium hexamethyl disilazide, sodium hexamethyldisilazide, potassium hexamethyldisilazide, sodium hydride and potassium hydride. A preferred base is lithium hexamethyl disilazide.

The dealkylating agent used in process step (e) for the dealkylation of the compound of Formula IXA may be selected from: pyridine hydrochloride, boron tribromide, boron trifluoride etherate and aluminium trichloride. A preferred dealkylating agent is pyridine hydrochloride.

Preparation of the starting compound of Formula VIA involves reacting 1-methyl-4-piperidone with a solution of 1,3,5-trimethoxybenzene in glacial acetic acid, to yield 1-methyl-4-(2,4,6-trimethoxyphenyl)-1,2,3,6-tetrahydropyridine, which is reacted with boron trifluoride diethyl etherate, sodium borohydride and tetrahydrofuran to yield 1-methyl-4-(2,4,6-trimethoxyphenyl)piperidin-3-ol. Conversion of 1-methyl-4-(2,4,6-trimethoxyphenyl)piperidin-3-ol to the compound of Formula VIA involves converting the hydroxyl group present on the piperidine ring of the compound, 1-methyl-4-(2,4,6-trimethoxyphenyl)piperidin-3-ol to a leaving group such as tosyl, mesyl, triflate or halide by treatment with an appropriate reagent such as p-toluenesulfonylchloride, methanesulfonylchloride, triflic anhydride or phosphorous pentachloride in the presence of oxygen nucleophiles such as triethylamine, pyridine, potassium carbonate or sodium carbonate, followed by ring contraction in the presence of oxygen nucleophiles such as sodium acetate or potassium acetate in an alcoholic solvent such as isopropanol, ethanol or propanol.

In an embodiment the CDK inhibitor is a compound of formula I wherein the phenyl group is substituted by 1, 2, or 3 identical or different substituents selected from: halogen selected from chlorine, bromine, fluorine or iodine; C₁-C₄-alkyl and trifluoromethyl.

In another embodiment, the CDK inhibitor is a compound of formula I wherein the phenyl group is substituted by 1, 2, or 3 halogens selected from chlorine, bromine, fluorine or iodine.

In another embodiment, the CDK inhibitor is a compound of formula I wherein the phenyl group is substituted by chlorine.

In a further embodiment, the CDK inhibitor represented by compound of formula I is (+)-trans-2-(2-Chloro-phenyl)-5,7-dihydroxy-8-(2-hydroxymethyl-1-methyl-pyrrolidin-3-yl)-chromen-4-one or its pharmaceutically acceptable salt.

In a still further embodiment, the CDK inhibitor represented by compound of formula I is (+)-trans-2-(2-Chloro-phenyl)-5,7-dihydroxy-8-(2-hydroxymethyl-1-methyl-pyrrolidin-3-yl)-chromen-4-one hydrochloride (designated herein as compound A).

In another embodiment, the CDK inhibitor is a compound of formula I wherein the phenyl group is disubstituted with a chloro and a trifluoromethyl group.

In a further embodiment, the CDK inhibitor represented by compound of formula I is (+)-trans-2-(2-Chloro-4-trifluoromethylphenyl)-5,7-dihydroxy-8-(2-hydroxymethyl-1-methyl-pyrrolidin-3-yl)-chromen-4-one; or its pharmaceutically acceptable salt.

In a still further embodiment, the CDK inhibitor represented by compound of formula I is (+)-trans-2-(2-Chloro-4-trifluoromethylphenyl)-5,7-dihydroxy-8-(2-hydroxymethyl-1-methyl-pyrrolidin-3-yl)-chromen-4-one hydrochloride (designated herein as compound B).

In an embodiment, the CDK inhibitor represented by a compound of formula I is an antiangiogenic agent.

In an embodiment, the CDK inhibitor represented by a compound of formula I is a HIF-1α inhibitor. In an embodiment, the CDK inhibitor represented by a compound of formula I is a VEG-F inhibitor. In an embodiment, the CDK inhibitor represented by a compound of formula I is a PARP enzyme inhibitor.

The manufacture of the compounds of formula I, which may be in the form of pharmaceutically acceptable salts, and the manufacture of oral and/or parenteral pharmaceutical composition containing the above compounds are disclosed in PCT Publication No. WO2004004632 (corresponding to U.S. Pat. No. 7,272,193) and PCT Publication No. WO2007148158. These PCT Publications disclose that the CDK inhibitors represented by formula I inhibit proliferation of many cancer cells. As indicated herein above the CDK inhibitors of formula I may be used in the form of their salts. Preferred salts of the compounds of formula I include hydrochloride salt, methanesulfonic acid salt and trifluoroacetic acid salt.

The compounds of formula I contain at least two chiral centers and hence exist in the form of two different optical isomers (i.e. (+) or (−) enantiomers). All such enantiomers and mixtures thereof including racemic mixtures are included within the scope of the invention. The enantiomers of the compound of formula I can be obtained as described above, by methods disclosed in PCT Publication Nos. WO2004004632, WO2008007169 and WO2007148158 or the enantiomers of the compound of formula I can also be obtained by methods well known in the art, such as chiral HPLC and enzymatic resolution. The term “enantiomerically pure” describes a compound which is present in an enantiomeric excess (ee) of greater than 95%. In another embodiment, the enantiomeric excess is greater than 97%. In still another embodiment, the enantiomeric excess is greater than 99%. The term “enantiomeric excess” describes the difference between the amount of one enantiomer and the amount of another enantiomer that is present in the product mixture.

Alternatively, the enantiomers of the compounds of formula I can be synthesized by using optically active starting materials. Thus, the definition of the compounds of formula I is inclusive of all possible stereoisomers and their mixtures. The definition of the compounds of formula I includes the racemic forms and the isolated optical isomers having the specified activity.

Paclitaxel, a cytotoxic antineoplastic agent comprised in the pharmaceutical combination of the present invention, is a natural diterpene product isolated from the Pacific yew tree Taxus brevifolia (Rowinsky et. al., J. Natl. Cancer Inst., 82, 1247-1259 (1990)). Isolation of paclitaxel and its structure is disclosed in J. Am. Chem. Soc. 93, 2325 (1971). It is an antimicrotubule agent that promotes the assembly of microtubules from tubulin dimers and stabilizes microtubules by preventing depolymerization. Paclitaxel is used to treat patients with lung, ovarian, breast cancer, head and neck cancer, and advanced forms of Kaposi's sarcoma. Paclitaxel has been approved for clinical use in the treatment of ovarian cancer (Merkman et al.; Yale Journal Of Biology and Medicine, 64:583, 1991) and for the treatment of breast cancer (Holmes et al; J. Nat. cancer Inst., 83; 1797, 1991), however, it is also useful in treating other cancers for example, it has been considered as a potential candidate for the treatment of head and neck cancer (Forastire et. al., Sem. Oncol., 20: 56, 1990) and lung cancer (M. Ghaemmaghami et al; Chest; 113; 86-91 (1998)). Paclitaxel is disclosed in U.S. Pat. No. 5,670,537 which is incorporated herein by reference for its teaching on the use or administration of paclitaxel in the treatment of susceptible cancers. Paclitaxel is commercially available as an injectable solution, Taxol®. A formulation in which paclitaxel is bound to albumin is sold under the trademark, Abraxane® (Abraxis BioScience, Inc.).

The general terms used hereinbefore and hereinafter preferably have the following meanings within the context of this disclosure, unless otherwise indicated:

As used herein, the term “combination” or “pharmaceutical combination”, means the combined administration of the anticancer agents namely paclitaxel and the CDK inhibitor (the compound of formula I); which anti-cancer agents may be administered independently at the same time or separately within time intervals that especially allow that the combination partners show a synergistic effect.

As used herein, the term “synergistic” means that the effect achieved with the methods and combinations of this invention is greater than the sum of the effects that result from using paclitaxel or a pharmaceutically acceptable salt thereof, and a CDK inhibitor, the compound of formula I or a pharmaceutically acceptable salt thereof, separately. Advantageously, such synergy provides greater efficacy at the same doses, and/or prevents or delays the build-up of multi-drug resistance.

A “therapeutically effective amount”, in reference to the treatment of triple negative breast cancer, refers to an amount capable of invoking one or more of the following effects in a subject receiving the combination of the present invention: (i) inhibition, to some extent, of tumor growth, including, slowing down and complete growth arrest; (ii) reduction in the number of cancerous cells; (iii) reduction in tumor size; (iv) inhibition (i.e., reduction, slowing down or complete stopping) of tumor cell infiltration into peripheral organs; (v) inhibition (i.e., reduction, slowing down or complete stopping) of metastasis; (vi) enhancement of anti-tumor immune response, which may, but does not have to, result in the regression or rejection of the tumor; and/or (vii) relief, to some extent, of one or more symptoms associated with triple negative breast cancer.

As used herein, the terms “manage”, “managing” and “management” refer to the beneficial effects that a subject or a patient derives from the pharmaceutical combination of the present invention when administered to said patient or subject so as to prevent the progression or worsening of TNBC.

As used herein the term “triple negative breast cancer(s)” or “TNBC” encompasses carcinomas of differing histopathological phenotypes. For example, certain TNBC are classified as “basal-like” (“BL”), in which the neoplastic cells express genes usually found in normal basal/myoepithelial cells of the breast, such as high molecular weight basal cytokeratins (CK, CK5/6, CK14, CK17), vimentin, p-cadherin, ccB crystallin, fascin and caveolins 1 and 2. Certain other TNBC, however, have a different histopathological phenotype, examples of which include high grade invasive ductal carcinoma of no special type, metaplastic carcinomas, medullary carcinomas and salivary gland-like tumors of the breast. The TNBC for the treatment of which the pharmaceutical combination of the present invention is provided may be non-responsive or refractory TNBC.

The term “non-responsive/refractory” as used herein, is used to describe subjects or patients having triple negative breast cancer(TNBC) having been treated with currently available cancer therapies for the treatment of TNBC such as chemotherapy, radiation therapy, surgery, hormonal therapy and/or biological therapy/immunotherapy wherein the therapy is not clinically adequate to treat the patients such that these patients need additional effective therapy, e.g., remain unsusceptible to therapy. The phrase can also describe subjects or patients who respond to therapy yet suffer from side effects, relapse, develop resistance, etc. In various embodiments, “non-responsive/refractory” means that at least some significant portions of the cancer cells are not killed or their cell division arrested. The determination of whether the cancer cells are “non-responsive/refractory” can be made either in vivo or in vitro by any method known in the art for assaying the effectiveness of treatment on cancer cells, using the art-accepted meanings of “refractory” in such a context. A cancer is “non-responsive/refractory” where the number of cancer cells has not been significantly reduced, or has increased.

As used herein the term “treatment cycle” refers to a time period during which a recurring sequence of administration of paclitaxel or a pharmaceutically acceptable salt thereof, and a CDK inhibitor of the compound of formula I or a pharmaceutically acceptable salt thereof, is carried out.

The term “apoptosis” refers to a type of cell death in which a series of molecular steps in a cell leads to its death. This is the body's normal way of getting rid of unneeded or abnormal cells. The process of apoptosis may be blocked in cancer cells. Also called programmed cell death. (Dictionary of cancer terms, National Cancer Institute)

As used herein the term “increasing apoptosis” is defined as an increase in the rate of programmed cell death, i.e. more cells are induced into the death process as compared to exposure (contact) with either the antineoplastic agent alone or the CDK inhibitor alone.

The term “subject” as used herein, refers to an animal, preferably a mammal, most preferably a human, who has been the object of treatment, observation or experiment.

In one embodiment, the present invention relates to a method for the treatment of triple negative breast cancer in a subject comprising administering to the subject a therapeutically effective amount of paclitaxel or its pharmaceutically acceptable salt and a cyclin dependent kinase (CDK) inhibitor selected from the compounds of formula I (as described herein) or a pharmaceutically acceptable salt thereof.

Accordingly, in the method of the present invention, triple negative breast cancer is treated in a subject by administering to the subject in need thereof, a therapeutically effective amount of paclitaxel or its pharmaceutically acceptable salt, in combination with a therapeutically effective amount of a CDK inhibitor selected from the compounds of formula I or a pharmaceutically acceptable salt thereof, wherein a synergistic effect results.

In an embodiment, the present invention relates to a method of treating triple negative breast cancer in a subject comprising administering to the subject a therapeutically effective amount of paclitaxel or its pharmaceutically acceptable salt and a therapeutically effective amount of a CDK inhibitor selected from the compounds of formula I or a pharmaceutically acceptable salt thereof, wherein paclitaxel and said CDK inhibitor are administered sequentially.

In an embodiment, the present invention relates to a method of treating triple negative breast cancer in a subject comprising administering to the subject a therapeutically effective amount of paclitaxel or its pharmaceutically acceptable salt and a therapeutically effective amount of the CDK inhibitor selected from the compounds of formula I or a pharmaceutically acceptable salt thereof, wherein paclitaxel is administered prior to the administration of said CDK inhibitor.

In an embodiment, the method of treating triple negative breast cancer of the present invention comprises administering paclitaxel and the CDK inhibitor in the dose range described herein.

In an embodiment, the present invention relates to a method of treating triple negative breast cancer in a subject comprising administering to the subject a therapeutically effective amount of paclitaxel or its pharmaceutically acceptable salt and a therapeutically effective amount of the CDK inhibitor selected from the compound A or compound B.

In an embodiment, the present invention relates to a method of treating triple negative breast cancer in a subject comprising administering to the subject a therapeutically effective amount of paclitaxel or its pharmaceutically acceptable salt and a therapeutically effective amount of the CDK inhibitor selected from the compound A or compound B, wherein paclitaxel and said compound A or compound B are administered sequentially.

In an embodiment, the present invention relates to a method of treating triple negative breast cancer in a subject comprising administering to the subject a therapeutically effective amount of paclitaxel or its pharmaceutically acceptable salt and a therapeutically effective amount of the CDK inhibitor selected from the compound A or compound B, wherein paclitaxel is administered prior to the administration of the compound A or compound B.

In an embodiment, the present invention relates to a pharmaceutical combination for use in the treatment of triple negative breast cancer, wherein said pharmaceutical combination comprises a therapeutically effective amount of paclitaxel or its pharmaceutically acceptable salt and a therapeutically effective amount of the CDK inhibitor selected from the compounds of formula I or a pharmaceutically acceptable salt thereof.

In an embodiment, the present invention relates to a pharmaceutical combination for use in the treatment of triple negative breast cancer, wherein said pharmaceutical combination comprises a therapeutically effective amount of paclitaxel or its pharmaceutically acceptable salt and a therapeutically effective amount of the CDK inhibitor selected from the compounds of formula I or a pharmaceutically acceptable salt thereof, wherein paclitaxel and said CDK inhibitor are administered sequentially.

In an embodiment, the present invention relates to a pharmaceutical combination for use in the treatment of triple negative breast cancer, wherein said pharmaceutical combination comprises a therapeutically effectively amount of paclitaxel or its pharmaceutically acceptable salt and a therapeutically effective amount of the CDK inhibitor selected from the compounds of formula I or a pharmaceutically acceptable salt thereof, wherein paclitaxel is administered prior to the administration of the CDK inhibitor.

In an embodiment, the present invention relates to the use of a pharmaceutical combination for the manufacture of a medicament for use in the treatment of triple negative breast cancer, wherein said pharmaceutical combination comprises a therapeutically effective amount of paclitaxel or its pharmaceutically acceptable salt and a therapeutically effective amount of the CDK inhibitor represented by a compound of formula I or a pharmaceutically acceptable salt thereof.

In an embodiment, the CDK inhibitor comprised in the pharmaceutical combination provided for use in the treatment of triple negative breast cancer, is selected from the compound A or compound B.

In an embodiment, the CDK inhibitor comprised in the pharmaceutical combination is the compound A.

In an embodiment, the CDK inhibitor comprised in the pharmaceutical combination is the compound B.

In an embodiment, the anticancer agents comprised in the pharmaceutical combination of the present invention may require different routes of administration, because of their different physical and chemical characteristics. For example, the CDK inhibitors of Formula I may be administered either orally or parenterally to generate and maintain good blood levels thereof, while the antineoplastic agent may be administered parenterally, by intravenous, subcutaneous or intramuscular route.

For oral use, the CDK inhibitors of formula I may be administered, for example, in the form of tablets or capsules, powders, dispersible granules, or cachets, or as aqueous solutions or suspensions. In the case of tablets for oral use, carriers which are commonly used include lactose, corn starch, magnesium carbonate, talc, and sugar, and lubricating agents such as magnesium stearate are commonly added. For oral administration in capsule form, useful carriers include lactose, corn starch, magnesium carbonate, talc and sugar.

For intramuscular, intraperitoneal, subcutaneous and intravenous use, sterile solutions of the active ingredient (paclitaxel or the CDK inhibitor) are usually employed, and the pH of the solutions should be suitably adjusted and buffered.

In an embodiment, the sterile solutions of the active ingredient used are prepared in saline or distilled water. The actual dosage of the active ingredients i.e. the anticancer agents contained in the combination may be varied depending upon the requirements of the patient and the severity of the condition being treated. Generally, treatment is initiated with smaller doses, which are less than the optimum dose of the compound. Thereafter, the dose of each ingredient is increased by small amounts until the optimum effect under the circumstances is reached. However, the amount of each ingredient in the pharmaceutical combination will typically be less than an amount that would produce a therapeutic effect if administered alone. For convenience, the total daily dose may be divided and administered in portions during the day if desired. In an embodiment, paclitaxel or its pharmaceutically acceptable salt, and a CDK inhibitor selected from the compounds of formula I or a pharmaceutically acceptable salt thereof are administered sequentially in injectable forms, such that paclitaxel is administered in a synergistically effective dose ranging from 10 mg to 1000 mg each, and the CDK inhibitor is administered in a synergistically effective dose ranging from 5 mg/m²/day to 1000 mg/m²/day, particularly in a dose ranging from 9 mg/m²/day to about 259 mg/m²/day.

In an embodiment, the pharmaceutical combination provided for use in the treatment of triple negative breast cancer is administered to a subject in need thereof, for six to eight treatment cycles, particularly six treatment cycles; two consecutive treatment cycles comprising the following steps:
i) a single dose administration of the pharmaceutical combination of paclitaxel and Compound A on day one of the treatment cycle;
ii) from second day, administration of one dose per day of Compound A for four consecutive days;
iii) a two-day interval wherein no drug (anticancer agent) is administered;
iv) optional administration of Compound A for five consecutive days followed by two-day interval with no drug (anticancer agent) administration;
v) optionally repeating step iv); and
vi) repeating steps i) to v) as a second treatment cycle, after an interval of three weeks from the beginning of step i).

In an embodiment, the pharmaceutical combination is administered to a subject in need thereof, for two to six treatment cycles, before surgery or after surgery or partially before and partially after surgery.

The combinations provided by this invention have been evaluated in certain assay systems, and in several different administrative schedules in vitro. The experimental details are as provided herein below. The data presented herein clearly indicate that paclitaxel when combined with a CDK inhibitor selected from the compounds of formula I exhibit synergistic effect. It is clearly indicated that the anticancer agents when used in combination in the treatment of triple negative breast cancer increases apoptosis or cytotoxicity in proliferative cells than when the cells are treated with only the CDK inhibitor, the compound of formula I alone or paclitaxel alone.

The representative compound, the compound A used in the pharmacological assays refers to (+)-trans-2-(2-Chloro-phenyl)-5,7-dihydroxy-8-(2-hydroxymethyl-1-methyl-pyrrolidin-3-yl)-chromen-4-one hydrochloride and was one of the compounds disclosed in the published PCT Publication No. WO2004004632, incorporated herein by reference.

The synergistic effect of the combination of the present invention comprising paclitaxel and a CDK inhibitor is now explained in more detail with reference to preferred embodiments thereof. It is to be noted that these are provided only as examples and not intended to limit the invention.

The following abbreviations or terms are used herein:

### ATCC: American Type Culture Collection, USA

ATP: Adenosine triphosphate

### CHCl₃: Chloroform

CDCl₃: Deuteriated chloroform
CO₂: Carbon dioxide

### CoA: Coenzyme A (Sigma Aldrich, USA)

DCC: N,N′-dicyclohexyl carbodiimide
DBTA: Dibenzoyl tartaric acid

### DMAP: 4-Dimethylaminopyridine

### DMF: N,N-dimethylformamide

### DMSO: Dimethylsulfoxide

DNA: Deoxyribonucleic acid

### DTT: Dithiothreitol (Sigma Aldrich, USA)

EDTA: Ethylene diamine tetra acetic acid
EtOAc: Ethyl acetate
FBS: Fetal bovine serum (Gibco, USA)
FCS: Fetal calf serum (Gibco, USA)

### g: Gram

### h: Hour

HCl: Hydrochloric acid
IPA: Isopropyl alcohol
KBr: Potassium bromide

### Kg: Kilogram

### L: Litre

MgSO₄: Magnesium sulfate

### MeOH: Methanol

### Min: Minute(s)

### mL: Millilitre

### μL: Microlitre

### μM: Micromolar

mmol: Millimolar

### mol: Mole

Na₂CO₃: Sodium carbonate
Na₂SO₄: Sodium sulfate
NaBH₄: Sodium borohydride
NaOH: Sodium hydroxide

### NCl: National Cancer Institute, USA

### ° C.: Degree Centigrade

PARP: Poly (ADP-ribose) polymerase
PBS: Phosphate buffered saline (Sigma Aldrich, USA)
PI: Propidium iodide (Sigma Aldrich, USA)

### RPMI: Roswell Park Memorial Institute, USA

### SDS-PAGE: Sodium Dodecyl Sulphate-Polyacrylamide Gel Electrophoresis

TFA: Trifluoroacetic acid

### THF: Tetrahydrofuran

### Cell-lines (Source: ATCC, USA):

TNBC: Triple negative breast cancer
MCF-7: (HER low, ER+, PR+, BRCA+/−allelic loss) breast cancer cell-line
T47-D: (HER low, ER +, PR +) breast cancer cell-line
ZR-75-1: (HER low, ER +, PR +) breast cancer cell-line
MDA-MB-468: (HER−, ER−, PR−) triple negative breast cancer cell-line
MDA-MB-231: (HER−, ER−, PR−) triple negative breast cancer cell-line
MDA-MB-435-S: (HER−, ER−, PR−) triple negative breast cancer cell-line
MDA-MB-361: (HER−, ER−, PR−) triple negative breast cancer cell-line
HBL-100: (HER−, ER−, PR−) triple negative breast cancer cell-line
BT-549: (HER−, ER−, PR−) triple negative breast cancer cell-line
HUVEC: Human umbilical vein endothelial cells

### Cell-Lines (Source: NCI, USA):

U251 HRE: Genetically engineered glioblastoma cells
U251 pGL3: Genetically engineered glioblastoma cells

### Antibodies (Source: Cell Signaling Technology, USA):

Cyclin D1 (cell cycle protein)
Bcl-2 (anti-apoptotic protein)
CDK4 (cyclin dependent kinase-4)

### Rb (Retinoblastoma)

pRb Ser780 (phospho-retinoblastoma)
PAR (substrate of PARP enzyme)
PARP (Poly (ADP-ribose) polymerase)
β-actin (house-keeping protein and used as a loading control for Western blot analysis)
Incubation conditions for cell-lines: 37° C. and 5% CO₂

The invention is further described by the following non-limiting examples which further illustrate the invention, and are not intended, nor should they be interpreted to, limit the scope of the invention.

### EXAMPLES

### Example 1

### Preparation of (÷)-trans-2-(2-Chlorophenyl)-5,7-dihydroxy-8-(2-hydroxy methyl-1-methyl-pyrrolidin-3-yl)-chromen-4-one hydrochloride (Compound A)

Sodium hydride (50%, 0.54 g, 11.25 mmol) was added in portions to a solution of (−)-trans-1-[2-Hydroxy-3-(2-hydroxymethyl-1-methylpyrrolidin-3-yl)-4,6-dimethoxy phenyl)-ethanone (0.7 g., 2.2 mmol) in dry DMF (15 mL) at 0° C., under nitrogen atmosphere and with stirring. After 10 min., methyl 2-chlorobenzoate (1.15 g., 6.75 mmol) was added. The reaction mixture was stirred at 25° C. for 2 h. Methanol was added carefully below 20° C. The reaction mixture was poured over crushed ice (300 g), acidified with 1:1 HCl (pH 2) and extracted using EtOAc (2×100 mL). The aqueous layer was basified using a saturated Na₂CO₃ (pH 10) and extracted using CHCl₃ (3×200 mL). The organic layer was dried (anhydrous Na₂SO₄) and concentrated. To the residue, conc. HCl (25 mL) was added and stirred at room temperature for 2 h. The reaction mixture was poured over crushed ice (300 g) and made basic using a saturated aqueous Na₂CO₃ solution. The mixture was extracted using CHCl₃ (3×200 mL). The organic extract was washed with water, dried (anhydrous Na₂SO₄) and concentrated to obtain the compound, (+)-trans-2-(2-chloro-phenyl)-8-(2-hydroxymethyl-1-methyl-pyrrolidin-3-yl)-5,7-dimethoxy-chromen-4-one. [Yield: 0.67 g (64%); mp: 91-93° C.; [α]_{D}²⁵=+5.8° (c=0.7, methanol)]

Molten pyridine hydrochloride (4.1 g, 35.6 mmol) was added to (+)-trans-2-(2-chloro-phenyl)-8-(2-hydroxymethyl-1-methyl-pyrrolidin-3-yl)-5,7-dimethoxy-chromen-4-one (0.4 g, 0.9 mmol) and heated at 180° C. for 1.5 h. The reaction mixture was cooled to 25° C., diluted with MeOH (10 mL) and basified using Na₂CO₃ to pH 10. The mixture was filtered and the organic layer was concentrated. The residue was suspended in water (5 mL), stirred for 30 min., filtered and dried to obtain the compound, (+)-trans-2-(2-chloro-phenyl)-5,7-dihydroxy-8-(2-hydroxymethyl-1-methyl-pyrrolidin-3-yl)-chromen-4-one. [Yield: 0.25 g (70%)]

(+)-trans-2-(2-chloro-phenyl)-5,7-dihydroxy-8-(2-hydroxymethyl-1-methyl-pyrrolidin-3-yl)-chromen-4-one (0.2 g, 0.48 mmol) was suspended in IPA (5 mL) and 3.5% HCl (25 mL) was added. The suspension was heated to get a clear solution. The solution was cooled and solid filtered to obtain the compound, (+)-trans-2-(2-Chlorophenyl)-5,7-dihydroxy-8-(2-hydroxymethyl-1-methyl-pyrrolidin-3-yl)-chromen-4-one hydrochloride or Compound A.

Yield: 0.21 g (97%); mp: 188-192° C.; [α]_{D}²⁵=+21.3° (c=0.2, methanol);

### Example 2

### Preparation of (+)-trans-2-(2-Chloro-4-trifluoromethyl-phenyl)-5,7-dihydroxy-8-(2-hydroxy-methyl-1-methylpyrrolidin-3-yl)-chromen-4-one hydrochloride (Compound B)

A mixture of the compound of trans-1-[2-Hydroxy-3-(2-hydroxymethyl-1-methylpyrrolidin-3-yl)-4,6-dimethoxy phenyl)-ethanone (1.16 g, 3.2 mmol), 2-chloro-4-trifluoromethylbenzoic acid (0.88 g, 4 mmol), DCC (1.35 g, 6.5 mmol) and DMAP (0.4 g, 3.27 mmol) were dissolved in dichloromethane (50 mL) and stirred at room temperature for 12 h. The reaction mixture is cooled to 0° C., the precipitated dicyclohexylurea was filtered and the organic layer concentrated and residue purified by column chromatography with 1% methanol in chloroform and 0.01% ammonia as eluent to obtain the compound, (+)-trans-2-chloro-4-trifluoromethylbenzoic acid 2-(2-acetoxymethyl-1-methyl-pyrrolidin-3-yl)-6-acetyl-3,5-dimethoxyphenyl ester [Yield: 1.44 g (78.8%)].

To a solution of n-BuLi (15% solution in hexane, 2.2 mL, 5 mmol) in THF (10 mL), maintained at 0° C. under nitrogen atmosphere, hexamethyldisilazane (1.08 mL, 5.1 mmol) was added dropwise and stirred for 15 min. To this, a solution of (+)-trans-2-chloro-4-trifluoromethylbenzoic acid 2-(2-acetoxymethyl-1-methyl-pyrrolidin-3-yl)-6-acetyl-3,5-dimethoxyphenyl ester (1.44 g, 2.5 mmol) in THF (10 mL) was added dropwise, maintaining the temperature at 0° C. After the addition, the reaction was allowed to warm to room temperature and stirred for 2.5 h. The reaction mixture was acidified with dilute HCl, and basified with 10% sodium bicarbonate to pH 8 to 9. The aqueous layer was extracted with chloroform (3×25 mL). The organic layer was washed with water (25 mL), brine (25 mL) and dried over anhydrous Na₂SO₄. The organic layer was concentrated under reduced pressure and dried under vacuum to yield acetic acid 3-{3-[3-(2-chloro-4-trifluoromethyl-phenyl)-3-oxo-propionyl]-2-hydroxy-4,6-dimethoxy-phenyl}-1-methyl-pyrrolidin-2-ylmethyl ester as an oil (1.3 g, 90.2%). This ester was dissolved in conc. HCl (10 mL) and stirred for 3 h to effect cyclisation. At the end of 3 h, the reaction mixture was basified with solid NaHCO₃ to pH 8 to 9. The aqueous layer was extracted with chloroform (25×3 mL) and washed with water (25 mL) and brine (25 mL). The organic layer was dried over anhydrous Na₂SO₄, concentrated under reduced pressure and dried over vacuum. The residue was purified by column chromatography with 3% methanol in chloroform and 0.1% ammonia as eluent to yield the compound, (+)-trans-2-(2-chloro-4-trifluoromethylphenyl)-8-(2-hydroxymethyl-1-methylpyrrolidin-3-yl)-5,7-dimethoxy-chromen-4-one as a yellow solid. [Yield: 0.56 g (48.2%)]

A mixture of (+)-trans-2-(2-chloro-4-trifluoromethylphenyl)-8-(2-hydroxymethyl-1-methylpyrrolidin-3-yl)-5,7-dimethoxy-chromen-4-one (0.25 g, 0.5 mmol), pyridine hydrochloride (0.25 g, 2.16 mmol) and a catalytic amount of quinoline was heated at 180° C. for a period of 2.5 h. The reaction mixture was diluted with methanol (25 mL) and basified with solid Na₂CO₃ to pH 10. The reaction mixture was filtered, and washed with methanol. The organic layer was concentrated and the residue purified by column chromatography using 0.1% ammonia and 4.5% methanol in chloroform as eluent to yield the compound, (+)-trans-2-(2-chloro-4-trifluoromethylphenyl)-5,7-dihydroxy-8-(2-hydroxy-methyl-1-methylpyrrolidin-3-yl)-chromen-4-one, as a yellow solid. [Yield: 0.15 g (63.7%)]

(+)-trans-2-(2-chloro-4-trifluoromethylphenyl)-5,7-dihydroxy-8-(2-hydroxy-methyl-1-methylpyrrolidin-3-yl)-chromen-4-one (0.1 g, 0.2 mmol) was suspended in methanol (2 mL) and treated with ethereal HCl and the organic solvent evaporated to yield the compound, (+)-trans-2-(2-chloro-4-trifluoromethyl-phenyl)-5,7-dihydroxy-8-(2-hydroxymethyl-1-methyl-pyrrolidin-3-yl)-chromen-4-one hydrochloride. [Yield: 0.1 g (92.8%)]

### Pharmacological Assays

### Example 3

### Cytotoxicity Assay using Propidium Iodide (PI)

The propidium iodide fluorescence assay (PI) was carried out according to the procedure mentioned in Anticancer Drugs, 1995, 6, 522-32.

The assay was developed to characterize the in vitro growth of human tumor cell lines as well as to test the cytotoxic activity of the compounds. Propidium iodide (PI) was used as a dye, which penetrates only damaged cellular membranes. Intercalation complexes are formed by PI with double-stranded DNA, which effect an amplification of the fluorescence. After freezing the cells at −20° C. for 24 h, PI had access to total DNA leading to total cell population counts. Background readings were obtained from cell-free wells containing media and propidium iodide.

The human breast cancer cell lines (i.e. MCF-7, T47-D, ZR-75-1, MDA-MB-468, MDA-MB-231, MDA-MB-435-S, MDA-MB-361, HBL-100, BT-549) were seeded at a density of 1500-3000 cells/well in 180 μL of DMEM (Dulbecco's Modified Eagle's Medium, Gibco, USA) or RPMI 1460, along with 10% FCS in a 96-well plate and incubated for about 16 h to allow the cells to adhere. The cells were then treated with varying concentrations of Compound A (0.1 to 3 μM). The above procedure was repeated in three TNBC cell-lines (MDA-MB-231, MDA-MB-468 and BT-549) for varying concentrations of the Compound A, paclitaxel (Sigma Aldrich, USA) and Sunitinib (Sutent®, LC Laboratories, USA), i.e. the concentration range for Compound A was 0.1-3 μM, the concentration range for paclitaxel was 0.1-10 μM while for Sunitinib (Sutent®), the concentration range was 1-100 μM, for a total period of 48 h. The plates were incubated in humidified 5% CO₂ incubator at 37° C.±1° C. Control wells were treated with vehicle (DMSO). At the end of the incubation periods, the plates were assayed using PI cytotoxicity assay protocol. Percent cytoxicity was calculated at various drug concentrations and from the graph plotted the IC₅₀ values were determined. The results of this study are presented in Tables 1A and 1B.

TABLE 1A
Antiproliferative activity of the Compound A, Paclitaxel and Sunitinib
for TNBC

| | Cell lines (IC₅₀ μM) | | |
|---|---|---|---|
| Compounds | MDA-MB-231 | BT-549 | MDA-MB-468 |
| Compound A | 1.0 | 0.8 | 1.0 |
| Paclitaxel | 0.9 | NT | NT |
| Sunitinib | 7.8 | 18 | 10 |
| (Sutent ®) | | | |

NT = Not tested

Table 1A shows the IC₅₀ values in μM for the Compound A, Paclitaxel and Sunitinib (Sutent®) in MDA-MB-231, BT-549 and MDA-MB-468 determined by cytotoxicity assay done after 48 h of the compound treatment.

TABLE 1B
Antiproliferative potential (IC₅₀ in μM) of Compound A in various
breast cancer cell lines as measured by PI assay

| S. No. | Cell-line | IC₅₀ (μM) |
|---|---|---|
| 1 | MCF-7 (HER low, ER+, PR +) | 1 |
| 2 | T47-D (HER low, ER+, PR+) | 0.5 |
| 3 | ZR-75-1 (HER low, ER+, PR+) | 0.9 |
| 4 | MDA-MB-468 (HER−, ER−, PR−) | 1 |
| 5 | MDA-MB-231 (HER−, ER−, PR−) | 1.0 |
| 6 | MDA-MB-435-S (HER−, ER−, PR−) | 0.3 |
| 7 | MDA-MB-361 (HER−, ER−, PR−) | 0.75 |
| 8 | HBL-100 (HER−, ER−, PR−) | 0.51 |
| 9 | BT-549 (HER−, ER−, PR−) | 0.8 |

Table 1B shows that the Compound A was found to be efficaciously antiproliferative against all the breast cancer cell lines irrespective of the genetic markers with IC₅₀ ranging from 0.3 to 1.0 μM.

### Example 4

### Clonogenic Assay or Colony Forming Assay

MDA-MB-231, MDA-MB-468 and MCF-7 cell lines were seeded in RPMI 1460 with 10% FCS, at a density of 1500 cells/well in six well plates. After 24 h incubation, cells were treated with IC₁₀, IC₃₀ and IC₅₀ concentrations of Compound A (as determined by the procedure of Example 3) for a period of 48 h and the IC₁₀, IC₃₀ and IC₅₀ values are presented in Table 2. The medium was removed at the end of the treatment and incubated in fresh medium (without drug) for 14 days. After 14 days the medium was aspirated and colonies were fixed with methanol and acetic acid mixture in the proportion of 2:1, rinsed with water and the fixation procedure was repeated. The plates were dried and colonies stained with 0.1% crystal violet for 5 min. The wells were finally rinsed with water and dried.

TABLE 2

| | Compound A concentrations | | |
|---|---|---|---|
| | (in μM) used for colony assay | | |
| Cell lines | IC₁₀ | IC₃₀ | IC₅₀ |
| MDA-MB-231 | 0.45 | 0.75 | 1.0 |
| MDA-MB-468 | 0.25 | 0.5 | 1.0 |
| MCF-7 | 0.37 | 0.75 | 1.0 |

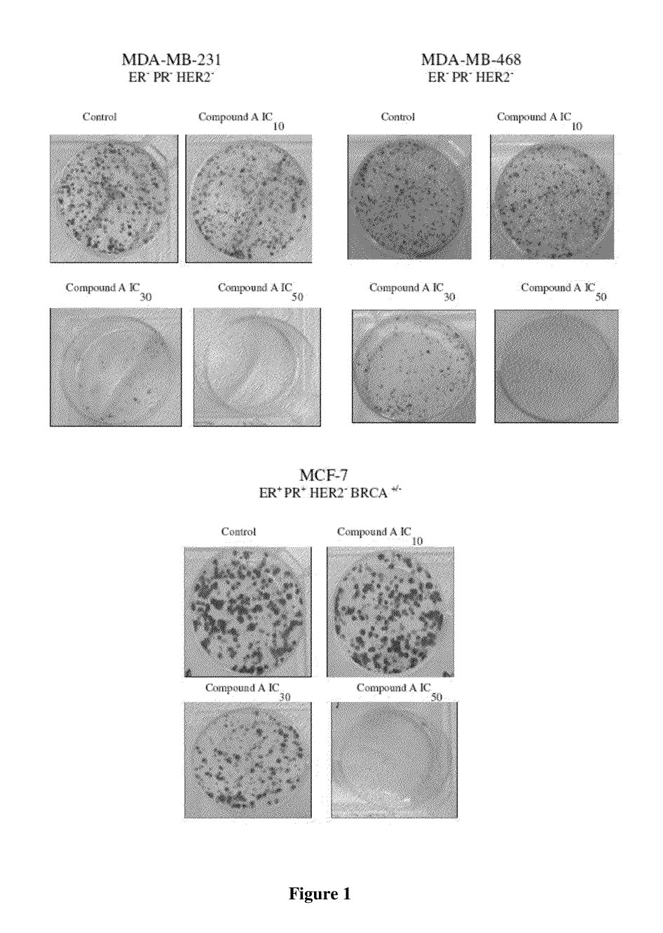

The results are depicted in FIG. 1, which shows the visual enhancement in the response by IC₁₀, IC₃₀ and IC₅₀ doses of Compound A, in MDA-MB-231, MDA-MB-468 and MCF-7 cell lines (Seeding density: 1500 cells/plate).

Compound A was found to inhibit the colony forming potential in a dose dependent manner.

### Example 5

### Effect of Compound A on Multicellular Tumor Spheroid (3D) Formation

The assay was carried out according to the method disclosed in Methods in Molecular Medicine, 2007, 140, 141-151.

The multicellular tumor spheroid (MCTS) model is one of the best-described 3D in vitro tumor model systems, which depicts many of the characteristics of tumor tissue and allows reproducible experiments, offering an excellent in vitro screening system. MCTS were propagated using the hanging drop method. Briefly, the cell monolayer was detached using trypsin-EDTA. Cell count was adjusted and 20 μL hanging droplets containing 1,000 cells/drop, were made in bacterial grade petridishes. These hanging drops were incubated for 24 h at 37° C. in a humidified atmosphere of 5% CO₂. The MCTS thus generated were cultured in the presence or absence of varying concentrations (0.3 μM to 30 μM) of Compound A for 72 h.

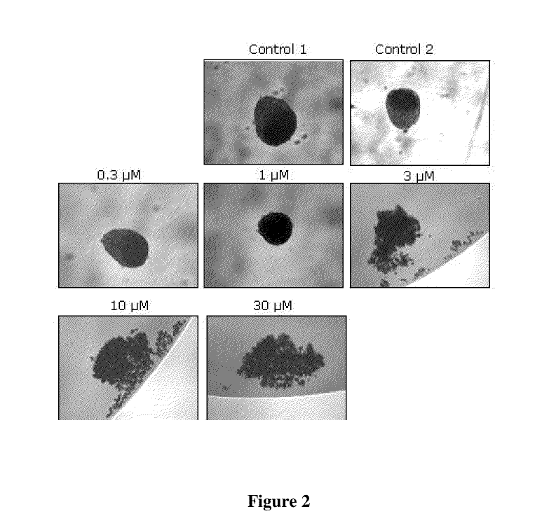

The results are presented in FIG. 2.

When MCF-7 cell suspension was co-incubated with varying concentrations of Compound A (0.3 μM to 30 μM) for propagation of MCTS, the spheroid formation was arrested from 3 μM concentration of Compound A onwards. The size of MCTS formed at 1 μM was also smaller as compared to control. This observation is important from the clinical point of view, as MCTS have been characterized sufficiently well to simulate the pathophysiological milieu in a patient tumor. Due to the gradient of oxygen in spheroids, which leads to formation of tumor hypoxia, it mimics the microenvironment prevailing in the tumor tissue. Effect of Compound A on spheroidal formation indicates that Compound A may be effective under hypoxia conditions.

### Example 6

### Time Dependent Effect of Compound a on Cell Cycle Progression and Apoptosis in MCF-7 (Her low, ER+, PR+, BRCA+/−Allelic Loss) and TNBC Cell-Line MDA-MB-231

Time dependent effect of Compound A on cell cycle progression and apoptosis was evaluated in two breast cancer cell lines. The asynchronous human breast cancer cell lines MCF-7 (Her low, ER+, PR+, BRCA+/−allelic loss) and MDA-MB-231 cells were seeded in 25 mm³ tissue culture flask at a density of 0.5×10⁶ cells per flask in RPMI 1460 with 10% FCS. After 24 h, cells were treated with 4.5 μM of Compound A for 0, 24, 48 and 72 h. Both detached and adherent cells were harvested (trypsinised) at different time points as mentioned in Table 3. After washing in phosphate buffered saline (PBS), cells were fixed in ice-cold 70% ethanol and stored at −20° C. until further analysis.

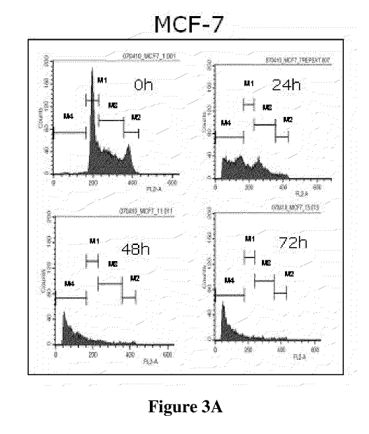

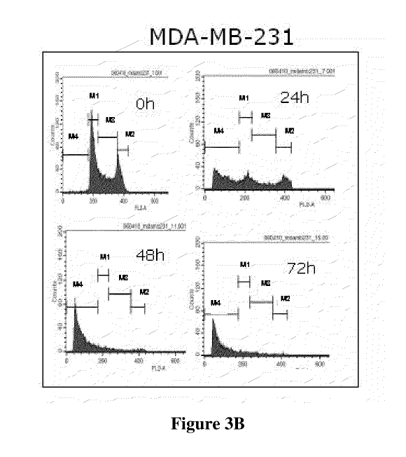

Before analysis, cells were washed twice with PBS to remove the fixative and re-suspended in PBS containing 50 μg/mL propidium iodide and 50 μg/mL RNaseA. After incubation at room temperature (20-35° C.) for 20 min, cells were analyzed using flow cytometry. A Becton Dickinson FACS Calibur flow cytometer (BD, USA) was used for these studies. The argon ion laser set at 488 nm was used as an excitation source. Cells with DNA content between 2n and 4n were designated as being in G1, S and G2/M phases of the cell cycle, as defined by the level of red fluorescence. Cells exhibiting less than 2n DNA content were designated as sub-G1 (apoptotic population) cells. The number of cells in each cell cycle compartment was expressed as a percentage of the total number of cells present. The results are shown in Table 3 and graphically presented in FIG. 3A (MCF-7 cell lines) and FIG. 3B (MDA-MB-231 cell lines).

TABLE 3
Percent apoptosis

% Apoptosis/Time points (h)

| Cell line | 0 | 24 | 48 | 72 |
|---|---|---|---|---|
| MDA-MB-231 | 1 | 45 | 85 | 85 |
| MCF-7 | 1 | 52 | 80 | 88 |

It is evident from the results shown in the above Table that compound A induced apoptosis in MCF-7 (Her low, ER+, PR+, BRCA+/−allelic loss) and TNBC cell-line MDA-MB-231. Maximum apoptosis was seen at 48 h and 72 h.

### Example 7

### Effect of Compound A in MCF-7 and MDA-MB-231 Cells using Western Blot Analysis

The Western blot assay was carried out according to the procedure disclosed in Molecular Cancer Therapeutics, 2007, 6, 918-925 with some modifications.

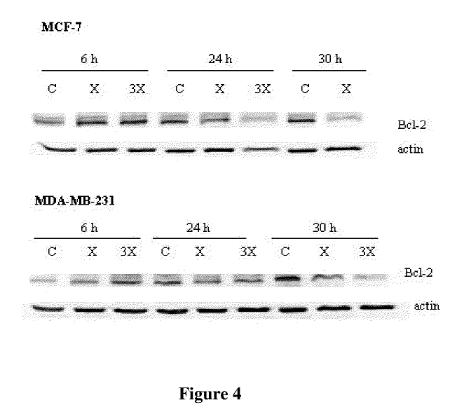

MCF-7 and MDA-MB-231 cells were seeded in RPMI 1460 medium with 10% FCS in 25 mm³ tissue culture flask and incubated for 24 h. The cells were treated with Compound A at 1.5 and 4.5 μM. At various time points, i.e. 6, 24 and 30 h the cells were harvested or trypsinized and lysed using lysis buffer (Sigma Aldrich, USA). Protein content was estimated. Lysate was applied to Sodium Dodecyl Sulphate-Polyacrylamide Gel Electrophoresis (SDS-PAGE) followed by western blotting (Molecular Cancer Therapeutics, 2007, 6, 918-925). Western blotting was done using specific antibodies to Bcl-2 and actin. The results are depicted in FIG. 4.

It can be seen from FIG. 4 that compound A down regulates antiapoptotic protein Bc1-2 in a dose dependent manner in both the cell lines. In MCF-7 cells, Bc1-2 is significantly down regulated from 24 h onwards, while in MDA-MB-231 significant down regulation was observed at 30 h.

### Example 8

### Effect of Compound a on Cell Cycle Progression and Apoptosis

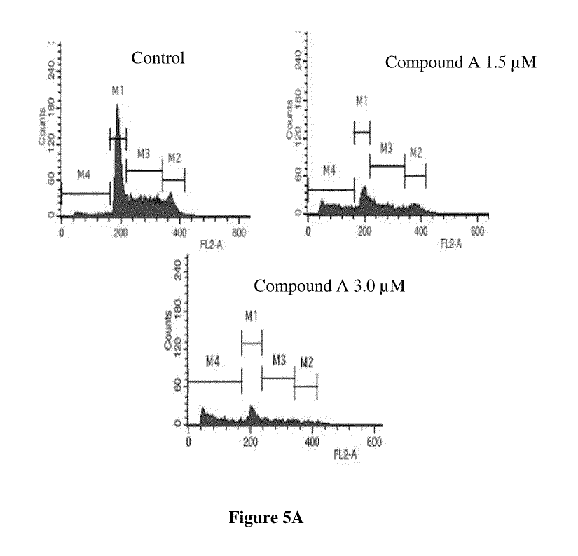

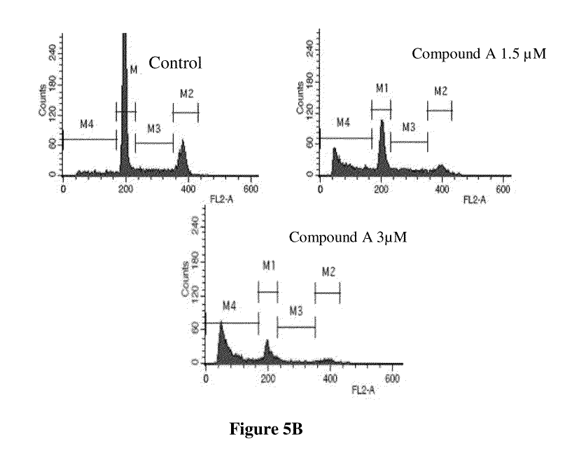

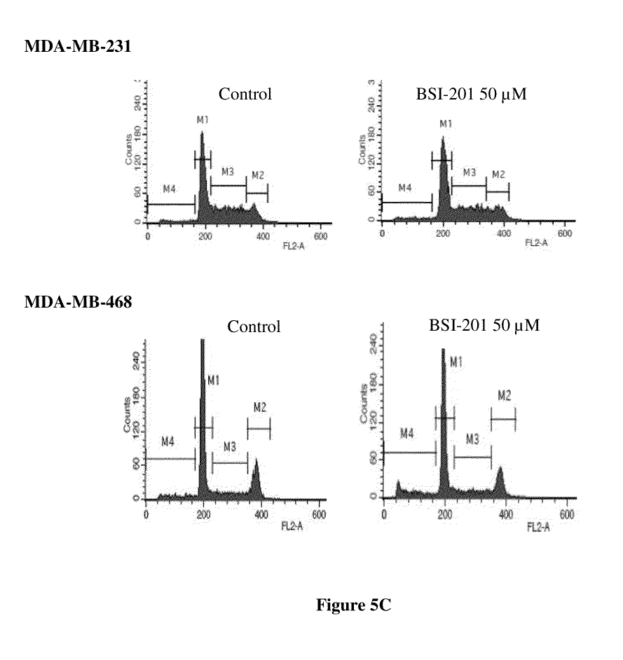

Comparison of the effect of Compound A and PARP inhibitor BSI-201 (Iniparib developed by Sanofi-Aventis. BSI-201 is prepared in-house) on cell cycle progression and apoptosis was evaluated in two TNBC cell lines. The asynchronous human TNBC cell lines MDA-MB-231 and MDA-MB-468 were seeded in 25 mm³ tissue culture flask at a density of 0.5×10⁶ cells per flask in RPMI 1460 with 10% FCS. After 24 h, cells were treated either with 1.5 and 3.0 μM of Compound A or 50 μM of PARP inhibitor BSI-201 for 72 h. After the incubation cells were harvested (trypsinised) and processed as given in example 6. The results are shown in Tables 4A and 4B; and graphically presented in FIGS. 5A, 5B and 5C.

TABLE 4A
Comparative analysis of percentage distribution of cells in different cell
cycle phases and apoptosis in MDA-MB-231 treated with
Compound A (a CDK inhibitor) and BSI-201 (a PARP inhibitor)

| | Concentrations | MDA-MB-231 |
|---|---|---|
| Compounds | μM | % apoptosis at 72 h |
| Control | — | 2.99 |
| Compound A | 1.5 | 27.45 |
| Compound A | 3 | 41.53 |
| BSI-201 | 50 | 3.96 |

TABLE 4B
Comparative analysis of percentage distribution of cells in different
cell cycle phases and apoptosis in MDA-MB-468 treated with
Compound A (a CDK inhibitor) and BSI-201 (a PARP inhibitor)

| | Concentrations | MDA-MB-468 |
|---|---|---|
| Compounds | μM | % apoptosis at 72 h |
| Control | — | 5.63 |
| Compound A | 1.5 | 36.09 |
| Compound A | 3 | 62.5 |
| BSI-201 | 50 | 12.67 |

The TNBC cell lines MDA-MB-231 and MDA-MB-468 showed dose dependent increase in apoptosis when treated with Compound A. BSI-201 (at 50 μM) showed no induction of apoptosis in MDA-MB-231. However, marginal apoptosis (12.67%) was observed in MDA-MB-468.

### Example 9

### Effect of Compound A on MCF-7 Cell Cycle Proteins and CDK4 Kinase Activity

### Step 1: Basal Level of Cyclin-D1 Expression

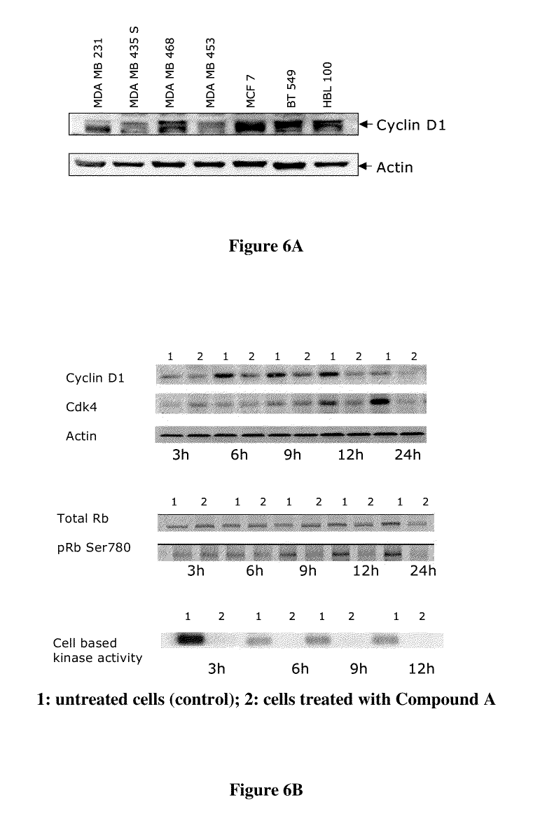

Basal level of cyclin-D1 expression was studied using western blot analysis (Molecular Cancer Therapeutics, 2007, 6, 918-925) across various breast cancer cell lines, i.e. MCF-7, MDA-MB-231, MDA-MB-468, MDA-MB-435 S, MDA-MB-453, BT-549 and HBL-100. These cells were seeded in RPMI 1460 medium with 10% FCS in 25 mm³ tissue culture flask and incubated for 24 h. The cells were harvested (trypsinised) and lysed using lysis buffer. Protein content was estimated. Lysate was applied to Sodium Dodecyl Sulphate-Polyacrylamide Gel Electrophoresis (SDS-PAGE) followed by Western blotting. Western blotting was done using cyclin D1 antibody and actin is used as a loading control. The results are shown in FIG. 6A. High cyclin D1 levels were observed in most of the breast cancer cell lines including triple negative breast cancer cell lines.

### Step 2: Effect of Compound A on MCF-7 Cell Cycle Proteins and CDK4 Kinase Activity

MCF-7 cells were seeded in RPMI 1460 medium with 10% FCS in 25 mm³ tissue culture flask and incubated for 24 h. These cells were treated with Compound A at 1.5 μM. At various time points viz. 3 h, 6 h, 9 h, 12 h and 24 h the cells were harvested (trypsinised) and lysed using lysis buffer. Protein content was estimated by Bradford method (Anal. Biochem., 1976, 72, 248-254). Lysate was applied to Sodium Dodecyl Sulphate-Polyacrylamide Gel Electrophoresis (SDS-PAGE) followed by Western blotting. Western blotting was done using specific antibodies to various cell cycle proteins viz. cyclin D1, CDK4, Rb and pRbSer780.

For immunoprecipitation assay, the MCF-7 cells were synchronized by serum starvation. These cells were treated with Compound A at 1.5 μM at various time points viz. 3 h, 6 h, 9 h and 12 h. Cells were harvested (trypsinised) and lysed using lysis buffer, and protein content was estimated. CDK4-D1 (Cyclin D1 and CDK4) was purified from the lysate by immunoprecipitation using specific antibody to CDK4. Immune complex was further purified using Protein A sepharose beads (Sigma Aldrich, USA). Immune complex was used to determine CDK4 activity using pRb as a substrate and ³²P labelled ATP (BRIT, India). Reaction mixed was applied to SDS-PAGE followed by transfer and autoradiography. The results are shown in FIG. 6B.

Compound A down regulates cyclin D1 and pRb in MCF-7 (Her low, ER+, PR+, BRCA+/−with allelic loss) in time dependent manner. Cyclin D1 and pRb expression show decrease from 6 h onwards and significantly at 12 h. There is no significant change in total Rb except at 24 h. Decrease in CDK4 kinase activity in cell-based assay was seen as early as 3 h onwards.

### Example 10

### Effect of Compound A on PARP Enzyme Activity as Measured by PAR Polymers

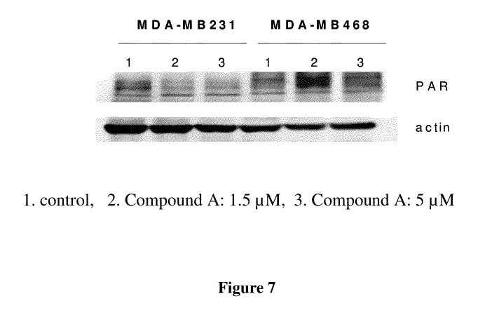

Poly(ADP-ribose) polymerase (PARP) is the principle member of a family of enzyme possessing poly(ADP-ribosylation) (PAR) catalytic capacity. To study PARP enzyme activity, PAR polymer formation was measured. MDA-MB-231 and MDA-MB-468 cells were seeded in RPMI 1460 medium with 10% FCS in 25 mm³ tissue culture flask and incubated for 24 h. These cells were treated at 1.5 μM and 5 μM of Compound A for 24 h. The cells were harvested (trypsinised) and lysed using lysis buffer. Western blotting (Molecular Cancer Therapeutics, 2007, 6, 918-925) was done with specific antibody to PAR. The results are shown in FIG. 7.

Compound A inhibits PARP enzyme activity as observed by the inhibition of PAR polymer formation in MDA-MB-231 cell line. However it was observed that in MDA-MB-468 the formation of PAR polymers is not inhibited.

### Example 11

### Effect of Compound A (24 h) on PARP and Cell Cycle Proteins in TNBC Cell Lines

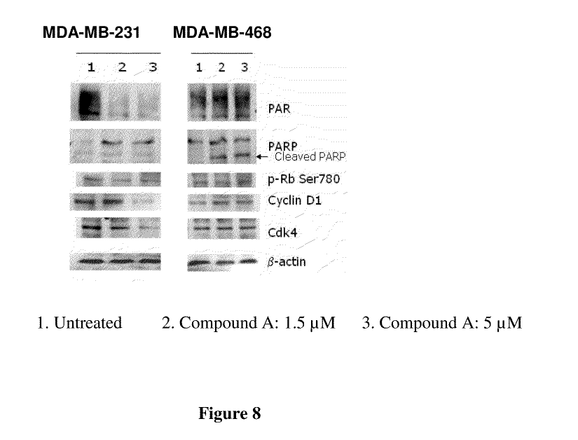

Correlation of PARP activity and cell cycle proteins cyclin D1, total Rb and pRb 780 were studied in two TNBC cell lines viz. MDA-MB-468 and MDA-MB-231. MDA-MB-231 and MDA-MB-468 cells were seeded in RPMI 1460 medium with 10% FCS in 25 mm³ tissue culture flask and incubated for 24 h. These cells were treated at 1.5 μM and 5 μM Compound A for 24 h. The cells were harvested (trypsinised) and lysed using lysis buffer. Western blotting was carried out (Molecular Cancer Therapeutically effectives, 2007, 6, 918-925) using specific antibody to PAR, PARP, cyclin D1, CDK4 and pRb Ser 780. The results are shown in FIG. 8.

In MDA-MB-231, Compound A inhibits PARP enzyme activity as seen by the inhibition of PAR polymer formation. This is accompanied by dose dependent decrease in pRb, cyclin D1 and CDK4. While in MDA-MB-468 although there was no change in the PAR polymer formation, PARP cleavage was prominent, which is an indication of apoptosis.

On treatment of TNBC cell line MDA-MB-231 with the compound A and incubation for 24 h, inhibition of PARP activity was observed in the cell line. However, MDA-MB-468 did not show PARP enzyme inhibition and instead showed cleaved PARP. Both of these are markers of cells undergoing apoptosis. Thus, it is evident that compound A induces significant apoptosis in both these cell lines.

### Example 12

### Effect of Compound A on HIF-1α Inhibition

### Test System in HIF-1α Reporter Gene Based Assay:

1) U251 HRE: The genetically engineered cells U251 HRE which stably express a recombinant vector in which the Luciferase reporter gene is under control of three copies of a canonical HRE.
2) U251 pGL3: A control cell line contains the firefly Luciferase reporter gene under control of the constitutively active SV40 promoter and enhancer that helps to exclude compounds that inhibit Luciferase expression in a nonspecific and/or HIF-1-independent fashion. These cells expressed high basal levels of Luciferase in normoxic conditions and slightly lower levels in hypoxic conditions.

U251 HRE cells were inoculated into 96 well white flat-bottomed plates at 10000-15000 cells/well in a volume of 180 μL and incubated for 24 h at 37° C., 5% CO₂, and ambient O₂. Compound A was tested at various concentrations viz. 0.01, 0.03, 0.1, 0.3, 1.0, 3.0 and 10 μM and plates were incubated for 20 h in a modular hypoxia chamber (Billups Rothenberg, MIC 101, USA) at 37° C., 5% CO₂, 1% O₂ and 94% N₂. After 20 h incubation, the plates were removed and incubated at room temperature, 5% CO₂, and ambient O₂ for 1.5 h. 40 μL of Bright Glo Luciferase reagent (Promega™, USA) was added and after 3 min, luminescence was measured using a Polar Star Plate Reader (USA) in luminescence mode. Appropriate control cells (U251 pGL3) were treated identically, except that they were treated at 37° C., 5% CO₂ and ambient O₂. Compound toxicity was assayed using the MTS assay.

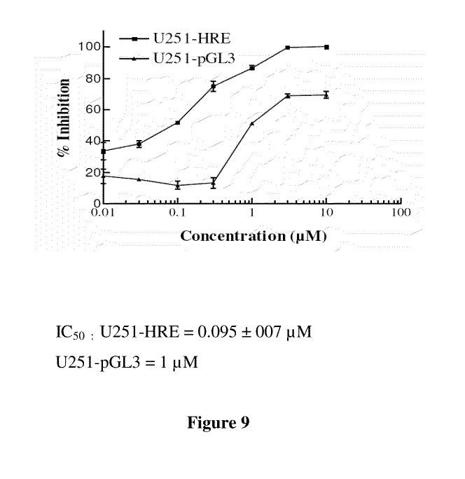

The results are graphically presented in FIG. 9.

Treatment with Compound A effectively blocked the expression of HIF-1α in a dose dependent manner in the U251 HRE cell line under hypoxia (<1% O₂). These compounds did not inhibit the luciferase expression in the control cell line U251 pGL3 under normoxia. This indicates that Compound A inhibits HIF-1α specifically.

### Example 13

### Effect of Compound A on VEGF Inhibition

A cell line M-9 is MDA-MB-231 which is stably co-transfected with the VEGF-Luc construct (VEGF promoter in pGL2-basic) and a plasmid containing the Geneticin (G418) resistance gene which forms VEGF promoter reporter gene. The expression of the reporter gene in the clone cells, as measured by luciferase activity, is stable.

The effect of Compound A on VEGF inhibition was evaluated using the VEGF reporter gene based assay.

### Reagents for VEGF Assay:

Lysis Assay Buffer (1×)
Tris-Phosphate (pH 7.8)-125 mM, DTT-10 mM, EDTA-10 mM, Glycerol-50% and Triton X-100-5%.

Luciferase Assay Reagent (LAR)
Luciferase Assay Buffer-8 mL, 530 μM ATP-530 μL, 270 μM CoA-1 mL and 170 μM Luciferin-1 mL.

Luciferase Assay Buffer (LAB) (1×)
Tricin (pH 7.8)-20 mM, Magnesia Alba-1.07 mM, MgSO4-2.67 mM, EDTA-0.1 mM and DTT-33.3 mM.
   ATP Stock made in LAB=5.85 mg/mL
   CoA Stock made in LAB=2.1 mg/mL
   Luciferin Stock made in LAB=1.5 mg/mL

### Protocol for VEGF Assay:

1. M-9 cells were sub-cultured and maintained in RPMI-1640 Medium with supplement of 10% FBS, and 4 μL/ml G418 (Stock 100 mg/mL) in a humidified incubator at 37° C. and 5% CO₂.
2. Cells were seeded at density of 3×10⁴ cells/well in 180 μL volume in tissue culture grade 96 well white plates as well as transparent plates and allowed to adhere for 16-20 h in humidified CO₂ incubator (5% CO₂) at 37° C. A total of two sets of plates were made, as the incubation conditions are different.
3. Compound A, Sutent® and BSI-201 were diluted in medium serially so that final desired concentrations are achieved in the respective wells. (not more than 0.5% concentration of DMSO in the wells).
4. INCUBATION CONDITIONS: One set of plates are incubated under ambient atmospheric condition with 5% CO₂, referred hereafter as NORMOXIC/OXIC PLATE. While the other set of plate goes in anoxic condition where the Oxygen concentration is less the 1%, and 94% Nitrogen, 5% CO₂, and referred hereafter as HYPDXIC PLATE. Temperature of incubation is 37° C. and humidity greater than 75%.
5. After 20-24 h incubation under Hypoxic and Normoxic conditions, plates are taken out from the incubators, medium from all the wells are removed from white plate. Cells are given a rapid wash with 100-150 μL/well Phosphate Buffer Saline (PBS). Cells are lysed with 40-50 μL Lysis buffer for 20 min.
6. To all the wells, 100 μL Luciferase Assay Reagent (LAR) are added, plates are immediately read for Luminescence on TOPCOUNT™ (Packard, USA). The percentage inhibitions and Inhibitory Concentration (IC₅₀) or Effective Concentration (EC₅₀) are calculated in comparison with Control (untreated) values.
IC₅₀ Values (μM) for VEGF Inhibition under Hypoxia:

Compound A: 0.31 μM

Sutent: 15 μM

BSI-201: >100 μM

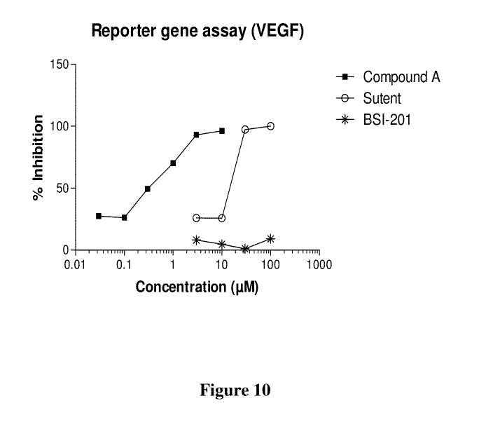

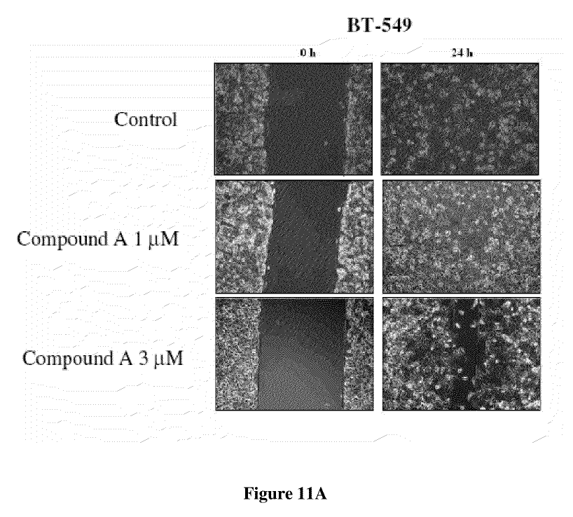

The results are graphically presented in FIG. 10.
Treatment with Compound A effectively blocked the expression of VEGF in a dose dependent manner.

### Example 14

### Effect of Compound A in Wound Healing Assay

The wound-healing assay is simple, inexpensive, and one of the earliest developed methods to study directional cell migration in vitro. This method mimics cell migration during wound healing in vivo.

Protocol:
1. MCF-7 cells were seeded in RPMI 1460 medium with 10% FCS in 25 mm³ tissue culture flask and incubated for 24 h.
2. The cells were trypsinized and seeded at a density of (0.5−2.0)×10⁶ per well in a sterile 6 well plate.
3. The plate was incubated for about 16 h in humidified CO₂ incubator (5% CO₂) at 37° C. under ambient oxygen levels. The cells were observed to form a confluent uniform monolayer on the complete surface of the well. The required number of cells for a confluent monolayer depends on both the particular cell type and size of dishes.
4. The cell monolayer in a straight line was scraped evenly to create a “scratch” with a pipette tip. The first image of the scratches was captured before addition of the compound.
5. Compound A was added at concentrations of 1 μM and 3 μM.
6. The plates were then kept in the incubator for further incubation. The time frame for incubation was determined empirically for the particular cell type used.
7. After the incubation, the dish was placed under a phase contrast microscope (Zeiss Axio Observer, Germany), reference point was matched, the photographed regions of the first image were aligned and the second image was captured. For each image distances between one side of the scratch and the other were measured.

Similar protocol was followed for BT-549 and MDA-MB-231 cell lines.

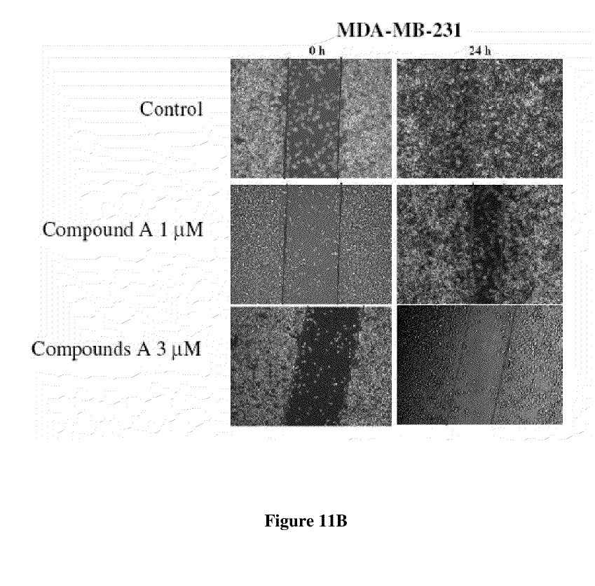

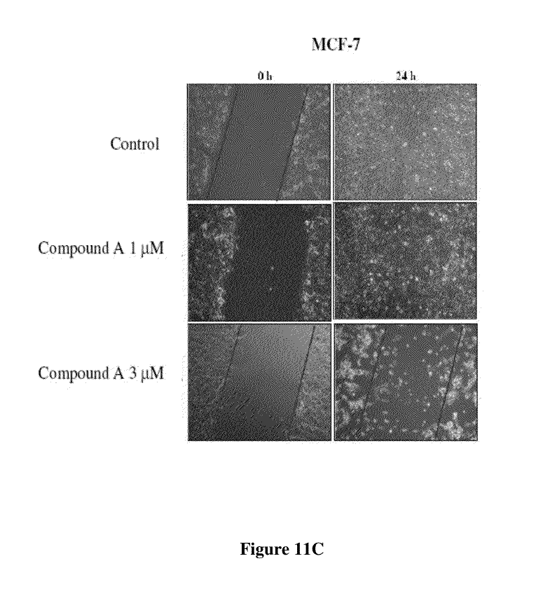

The results are presented in FIGS. 11A, 11B and 11C.

Compound A showed potent anti migratory effect in all the breast cancer cell lines including triple negative breast cancer cell line. The control cells showed complete healing after an incubation of 24 h. The cells treated with Compound A showed very less migration from both sides, thus indicating potent anti migratory effect.

### Example 15

### Angiogenesis of Compound A in Endothelial Tube Formation Assay

The Tube Formation Assay represents a simple but powerful model for studying inhibition and induction of angiogenesis. The assay relies on the endothelial cells' ability to form distinct blood-vessel like tubules in an extracellular matrix (BD Matrigel™ Matrix, USA) where they can subsequently be visualized by microscopy. It enables analysis of angiogenic tubules in a 3 dimensional matrix that better resembles the native physiological environment.

### Protocol

### Endothelial Cell Tube Formation Assay

Confluent HUVEC (Human umbilical vein endothelial cells) were cultured with above mentioned endothelial medium to desired confluence. For HUVEC 60-80% confluence is recommended.

Endothelial cell suspensions were prepared by trypsinizing the cell monolayers and resuspending the cells in culture medium with 5-10% serum. (0.5−1)×10⁶ cells per 180 μL of cell suspension were added (per well of 24 well plate) to the medium (BD Matrigel Matrix) which, had been thawed at 4° C. This suspension was then added to the plates and kept for incubation. The cells were allowed to adhere for 2-3 h and then Compound A (1 μM), Rotenone (1 μM) (Sigma-Aldrich, USA) and Topotecan (3 μM) (Sigma-Aldrich, USA) (20 μL of 10× stocks) were added to the respective wells. DMSO was used as the control. After 24-48 h of incubation the cells were observed under a phase contrast microscope (Zeiss Axio Observer, Germany) for tube formation and angiogenesis.

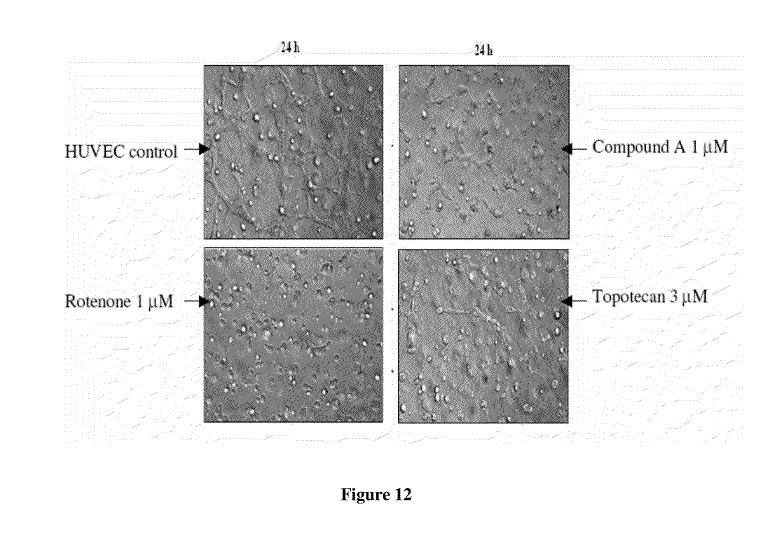

The results are shown in FIG. 12.

Compound A effectively inhibited endothelial tube formation and thus angiogenesis in the 3D gel HUVEC tube formation assay. Compound A at 1 μM was comparable to Rotenone (standard VEGF inhibitor) and better than Topotecan (known HIF-1α inhibitor in clinical trials).

### Example 16

### In vitro Cytotoxicity Assay

### Methods

### Effect of the Combination of Compound A and Paclitaxel on Triple Negative Breast Cancer Cell Line, MDA-MB-231 Using Propidium Iodide (PI) Assay

### Assay Protocol:

The propidium iodide fluorescence assay (PI) was carried out according to the procedure mentioned in Anticancer Drugs, 1995, 6, 522-32.

The assay was developed to characterize the in vitro growth of human tumor cell lines as well as to test the cytotoxic activity of the test compounds. Propidium iodide (PI) was used as a dye, which penetrates only, damaged cellular membranes. Intercalation complexes are formed by PI with double-stranded DNA, which effect an amplification of the fluorescence. After freezing the cells at −20° C. for 24 h, PI had access to total DNA leading to total cell population counts. Background readings were obtained from cell-free wells containing media and propidium iodide.

The human triple negative breast cancer cell line, MDA-MB-231 was seeded at a density of 1500-3000 cells/well in 180 μL of RPMI-1640 medium in a 96-well plate and incubated for about 16 h in humidified 5% CO₂ incubator at 37±1° C. to allow the cells to adhere. The cells were then treated according to the schedule for drug treatment presented in Table 5. The schedule consists of six treatment groups. In every treatment group, 20 μL of 10× compound (dissolved first in DMSO and then diluted in cell medium, final DMSO concentration not exceeding 0.5%) was used in the wells and the plate was incubated in humidified 5% CO₂ incubator at 37±1° C. The medium was removed from the wells and washed with PBS. 100 μL of PI working solution (7 μg/mL) per well was added and the plates were stored at −80° C. for about 16 h. The plates were thawed and the fluorescence was measured using the POLARstar optima plate reader (USA) at excitation 536 nm and emission 590 nm.

(PI stock solution of 1 mg/mL was prepared by dissolving 1 mg PI in 1 mL of distilled water. PI working solution was prepared by adding 140 μL of stock solution to PBS to make up the volume to 220 mL (7 μg/mL)).

Schedule: It consists of six treatment groups.
1) The MDA-MB-231 cells were treated with paclitaxel (0.03, 0.1, 0.3, 1.0 and 3.0 μM concentrations IC μM) and incubated for 24 h followed by removal of medium, addition of complete medium (CM) and incubation for 72 h (Group IA).
2) The cells were treated with complete medium and incubated for 24 h followed by removal of medium and addition of Compound A (IC₅₀=1 μM) and incubation for 72 h (Group IIA).
3) The cells were treated with complete medium and incubated for 24 h followed by removal of medium, addition of Sunitinib (Sutent®, IC₅₀=7.8 μM) and incubation for 72 h (Group IIIA)
4) The cells were treated with varying concentrations of paclitaxel (0.03, 0.1, 0.3, 1.0 and 3.0 μM) and incubated for 24 h followed by removal of medium, addition of Compound A (IC₅₀=1 μM) and incubation for 72 h (Group IVA).
5) The cells were treated with paclitaxel (0.03, 0.1, 0.3, 1.0 and 3.0 μM) and incubated for 24 h followed by removal of medium, addition of Sunitinib (Sutent®, IC₅₀=7.8 μM) and incubation for 72 h (Group VA).
6) The cells were treated with DMSO vehicle and incubated for 24 h followed by removal of medium, addition of complete medium (CM: medium+10% FCS) and incubation for 72 h (Group VIA).

The schedule of drug treatment is shown in Table 5.

TABLE 5

| | Schedule for drug treatment | |
|---|---|---|
| Group | At 0 h | At 24 h |
| IA | Paclitaxel (0.03 μM) and | CM and 72 h incubation |
| | 24 h incubation | |
| | Paclitaxel (0.1 μM) and | |
| | 24 h incubation | |
| | Paclitaxel (0.3 μM) and | |
| | 24 h incubation | |
| | Paclitaxel (1.0 μM) and | |
| | 24 h incubation | |
| | Paclitaxel (3.0 μM) and | |
| | 24 h incubation | |
| IIA | CM and 24 h incubation | Compound A (IC₅₀ = 1 μM) |
| | | and 72 h incubation |
| IIIA | CM and 24 h incubation | Sunitinib (Sutent ®, IC₅₀ = 7.8 μM) |
| | | and 72 h incubation |
| IVA | Paclitaxel (0.03 μM) and | Compound A (IC₅₀ = 1 μM) |
| | 24 h incubation | and 72 h incubation |
| | Paclitaxel (0.1 μM) and | |
| | 24 h incubation | |
| | Paclitaxel (0.3 μM) and | |
| | 24 h incubation | |
| | Paclitaxel (1.0 μM) and | |
| | 24 h incubation | |
| | Paclitaxel (3.0 μM) and | |
| | 24 h incubation | |
| VA | Paclitaxel (0.03 μM) and | Sunitinib (Sutent ®, IC₅₀ = 7.8 μM) |
| | 24 h incubation | and 72 h incubation |
| | Paclitaxel (0.1 μM) and | |
| | 24 h incubation | |
| | Paclitaxel (0.3 μM) and | |
| | 24 h incubation | |
| | Paclitaxel (1.0 μM) and | |
| | 24 h incubation | |
| | Paclitaxel (3.0 μM) and | |
| | 24 h incubation | |
| VIA | Vehicle control and | CM and 72 h incubation |
| | 24 h incubation | |

CM = Complete medium

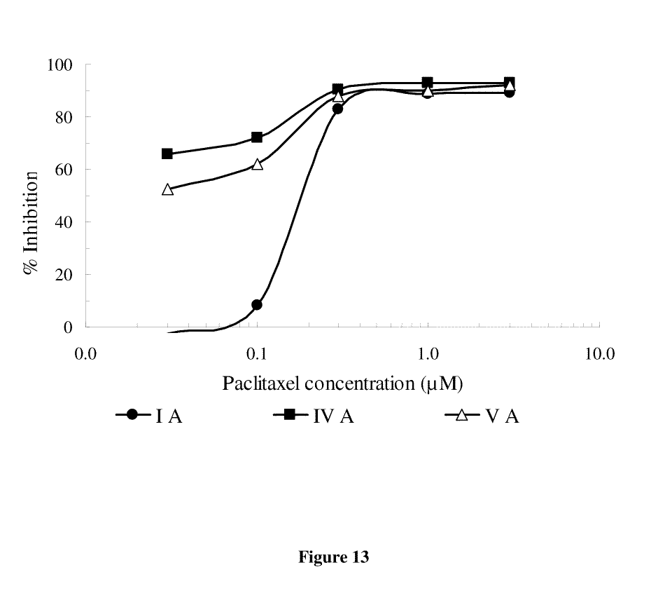

At the end of the incubation periods, the plate was assayed using the PI cytotoxicity assay protocol. The results are shown in Table 6, Table 7 and FIG. 13.

TABLE 6

| Concentration | % inhibition of | % inhibition of | % inhibition of |
|---|---|---|---|
| of paclitaxel (μM) | Group IA | Group IVA | Group VA |
| 3.0 | 89.1 | 92.8 | 92.1 |
| 1.0 | 88.7 | 92.7 | 90.1 |
| 0.3 | 82.9 | 90.3 | 88.0 |
| 0.1 | 8.3 | 72.3 | 62.1 |
| 0.03 | −2.6 | 65.9 | 52.5 |

Group IA (CM and 72 h incubation)
Group VIA (Compound A and 72 h incubation)
Group VA (Sunitinib and 72 h incubation)

The synergistic effects in TNBC MDA-MB-231 cell line have been evaluated using the CompuSyn software by Chou and Talalay, described in Pharmacological Reviews, 2006, 58, 621-681. Combination index (CI) is used to evaluate if a combination is additive, synergistic or antagonistic. CI<1 is synergistic, CI=1 is additive and CI>1 is antagonistic. The combination index as evaluated for the combination groups is shown in Table 7.

TABLE 7
CI values for combination groups in MDA-MB-231

| Group | CI |
|---|---|
| IVA | 0.4-0.8 |
| VA | 0.5-1.0 |

The combination of paclitaxel and Compound A was comparatively more synergistic than paclitaxel and Sutent® as is evident from the value of the combination index in the TNBC cell lines MDA-MB-231.

### Cytotoxicity Determination:

The IC₅₀ values in μM for Compound A, paclitaxel and Sunitinib (Sutent®) in MDA-MB-231, BT-549 and MDA-MB-468 determined by cytotoxicity assay done after 48 h of compound treatment as determined in Table 1A of Example 3 were used in Example 16. After completion of the compound treatment i.e. at the end of 48 h, the plates were processed for PI assay and the percent cytotoxicity was calculated as compared to DMSO (vehicle) control.

The results of the schedule used in the combination experiments indicated that Compound A is synergistic when used in combination with paclitaxel.

### Example 17

### In Vitro Cytotoxicity Assay

### Methods

Effect of the Combination of Compound A and Paclitaxel on Triple Negative Breast Cancer Cell Line, BT-549 using Propidium Iodide (PI) Assay

### Assay Protocol:

The propidium iodide fluorescence assay (PI) was carried out according to the procedure mentioned in Anticancer Drugs, 1995, 6, 522-32.

The assay was developed to characterize the in vitro growth of human tumor cell lines as well as to test the cytotoxic activity of the compounds. Propidium iodide (PI) was used as a dye, which penetrates only, damaged cellular membranes. Intercalation complexes are formed by PI with double-stranded DNA, which effect an amplification of the fluorescence. After freezing the cells at −20° C. for 24 h, PI had access to total DNA leading to total cell population counts. Background readings were obtained from cell-free wells containing media and propidium iodide.

The human triple negative breast cancer cell line, BT-549 was seeded at a density of 1500-3000 cells/well in 180 μL of RPMI-1640 medium in a 96-well plate and incubated for about 16 h in humidified 5% CO₂ incubator at 37±1° C. to allow the cells to adhere. The cells were then treated according to the schedule in Table 8. Each schedule consists of six treatment groups. In every treatment group, 20 μL of 10× compound (dissolved first in DMSO and then diluted in cell medium, final DMSO concentration not exceeding 0.5%) was used in the wells and the plate was incubated in humidified 5% CO₂ incubator at 37±1° C. The medium was removed from the wells and washed with PBS. 100 μL of PI working solution (7 μg/mL) per well was added and the plates were stored at −80° C. for about 16 h. The plates were thawed and the fluorescence was measured using the POLARstar optima plate reader (USA) at excitation 536 nm and emission 590 nm.

(PI stock solution of 1 mg/mL was prepared by dissolving 1 mg PI in 1 mL of distilled water. PI working solution was prepared by adding 140 μL of stock solution to PBS to make up the volume to 220 mL (7 μg/mL)).

Schedule: It consists of six treatment groups.
1) The BT-549 cells were treated with paclitaxel (0.03, 0.1, 0.3, 1.0 and 3.0 μM concentrations) and incubated for 24 h followed by removal of medium, addition of complete medium and incubation for 72 h (Group IB).
2) The cells were treated with complete medium and incubated for 24 h followed by removal of medium, addition of Compound A (IC₅₀=1 μM) and incubation for 72 h (Group IIB).
3) The cells were treated with complete medium and incubated for 24 h followed by removal of medium, addition of Sunitinib (Sutent®, IC₅₀=7.8 μM) and incubation for 72 h (Group IIIB).
4) The cells were treated with paclitaxel (0.03, 0.1, 0.3, 1.0 and 3.0 μM) and incubated for 24 h followed by removal of medium, addition of Compound A (IC₅₀=1 μM) and incubation for 72 h (Group IVB).
5) The cells were treated with paclitaxel (0.03, 0.1, 0.3, 1.0 and 3.0 μM) and incubated for 24 h followed by removal of medium, addition of Sunitinib (Sutent®, IC₅₀=7.8 μM) and incubation for 72 h (Group VB).
6) The cells were treated with DMSO vehicle and incubated for 24 h followed by removal of medium, addition of complete medium (CM: medium+10% FCS) and incubation for 72 h (Group VIB).

The schedule of drug treatment is shown in Table 8.

TABLE 8

| | Schedule for drug treatment | |
|---|---|---|
| Group | At 0 h | At 24 h |
| IB | Paclitaxel (0.03 μM) and | CM and 72 h incubation |
| | 24 h incubation | |
| | Paclitaxel (0.1 μM) and | |
| | 24 h incubation | |
| | Paclitaxel (0.3 μM) and | |
| | 24 h incubation | |
| | Paclitaxel (1.0 μM) and | |
| | 24 h incubation | |
| | Paclitaxel (3.0 μM) and | |
| | 24 h incubation | |
| IIB | CM and 24 h incubation | Compound A (IC₅₀ = 1 μM) |
| | | and 72 h incubation |
| IIIB | CM and 24 h incubation | Sunitinib (Sutent ®, IC₅₀ = 7.8 μM) |
| | | and 72 h incubation |
| IVB | Paclitaxel (0.03 μM) and | Compound A (IC₅₀ = 1 μM) |
| | 24 h incubation | and 72 h incubation |
| | Paclitaxel (0.1 μM) and | |
| | 24 h incubation | |
| | Paclitaxel (0.3 μM) and | |
| | 24 h incubation | |
| | Paclitaxel (1.0 μM) and | |
| | 24 h incubation | |
| | Paclitaxel (3.0 μM) and | |
| | 24 h incubation | |
| VB | Paclitaxel (0.03 μM) and | Sunitinib (Sutent ®, IC₅₀ = 7.8 μM) |
| | 24 h incubation | and 72 h incubation |
| | Paclitaxel (0.1 μM) and | |
| | 24 h incubation | |
| | Paclitaxel (0.3 μM) and | |
| | 24 h incubation | |
| | Paclitaxel (1.0 μM) and | |
| | 24 h incubation | |
| | Paclitaxel (3.0 μM) and | |
| | 24 h incubation | |
| VIB | Vehicle control and | CM and 72 h incubation |
| | 24 h incubation | |

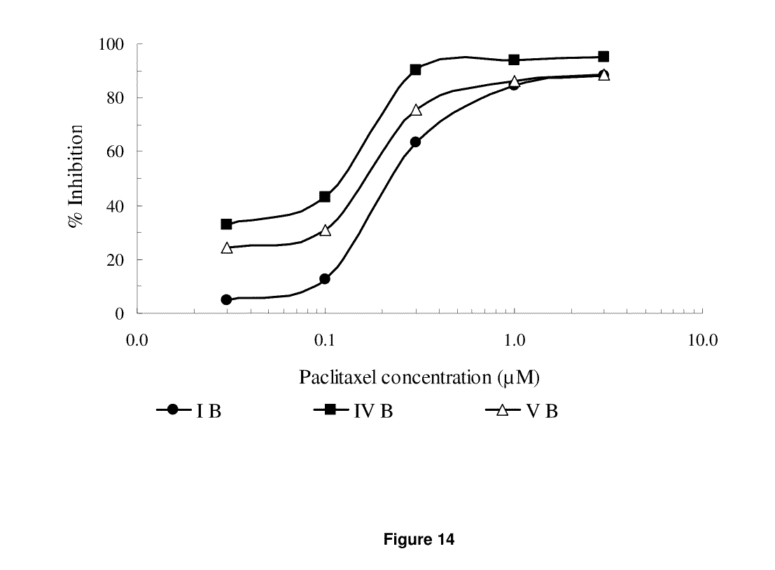

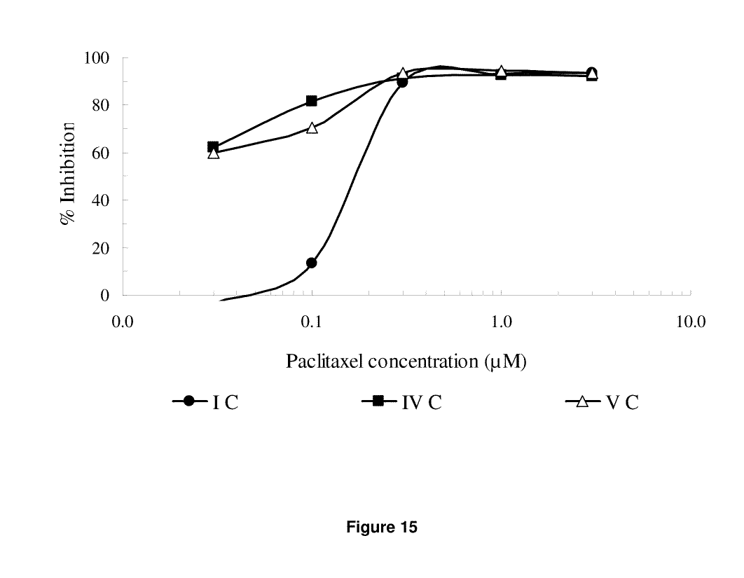

The combination index as evaluated for the combination groups is shown in Table 9. The results are shown in FIG. 14.

TABLE 9
CI values for combination groups in BT-549

| Group | CI |
|---|---|
| IVB | 0.4-0.8 |
| VB | >1.0 |

The combination of Paclitaxel and Compound A was comparatively more synergistic than Paclitaxel and Sutent as indicated by the combination index in the TNBC cell line BT-549.

### Example 18

### In Vitro Cytotoxicity Assay

### Methods

Effect of the Combination of Compound A and Paclitaxel on Triple Negative Breast Cancer Cell Line, MDA-MB-468 using Propidium Iodide (PI) Assay

### Assay Protocol:

The propidium iodide fluorescence assay (PI) was carried out according to the procedure mentioned in Anticancer Drugs, 1995, 6, 522-32.

The assay was developed to characterize the in vitro growth of human tumor cell lines as well as to test the cytotoxic activity of the compounds. Propidium iodide (PI) was used as a dye, which penetrates only, damaged cellular membranes. Intercalation complexes are formed by PI with double-stranded DNA, which effect an amplification of the fluorescence. After freezing the cells at −20° C. for 24 h, PI had access to total DNA leading to total cell population counts. Background readings were obtained from cell-free wells containing media and propidium iodide.

The human triple negative breast cancer cell line, MDA-MB-468 was seeded at a density of 1500-3000 cells/well in 180 μL of RPMI-1640 medium in a 96-well plate and incubated for about 16 h in humidified 5% CO₂ incubator at 37±1° C. to allow the cells to adhere. The cells were then treated according to the schedule in Table 10. Each schedule consists of six treatment groups. In every treatment group, 20 μL of 10× compound (dissolved first in DMSO and then diluted in cell medium, final DMSO concentration not exceeding 0.5%) was used in the wells and the plate was incubated in humidified 5% CO₂ incubator at 37±1° C. The medium was removed from the wells and washed with PBS. 100 μL of PI working solution (7 μg/mL) per well was added and the plates were stored at −80° C. for about 16 h. The plates were thawed and the fluorescence was measured using the POLARstar optima plate reader (USA) at excitation 536 nm and emission 590 nm.

(PI stock solution of 1 mg/mL was prepared by dissolving 1 mg PI in 1 mL of distilled water. PI working solution was prepared by adding 140 μL of stock solution to PBS to make up the volume to 220 mL (7 μg/mL)).

### Schedule:

It consists of six treatment groups.
1) The MDA-MB-468 cells were treated with paclitaxel (0.03, 0.1, 0.3, 1.0 and 3.0 μM concentrations) and incubated for 24 h followed by removal of medium, addition of complete medium and incubation for 72 h (Group IC).
2) The cells were treated with complete medium and incubated for 24 h followed by removal of medium, addition of Compound A (IC₅₀=1 μM) and incubation for 72 h (Group IIC).
3) The cells were treated with complete medium and incubated for 24 h followed by removal of medium, addition of Sunitinib (Sutent®, IC₅₀=7.8 μM) and incubation for 72 h (Group IIIC).
4) The cells were treated with paclitaxel (0.03, 0.1, 0.3, 1.0 and 3.0 μM) and incubated for 24 h followed by removal of medium, addition of Compound A (IC₅₀=1 μM) and incubation for 72 h (Group IVC).
5) The cells were treated with paclitaxel (0.03, 0.1, 0.3, 1.0 and 3.0 μM) and incubated for 24 h followed by removal of medium, addition of Sunitinib (Sutent®, IC₅₀=7.8 μM) and incubation for 72 h (Group VC).
6) The cells were treated with DMSO vehicle and incubated for 24 h followed by removal of medium, addition of complete medium (CM: medium+10% FCS) and incubation for 72 h (Group VIC).
   The schedule of drug treatment is shown in Table 10.

TABLE 10

| | Schedule for drug treatment | |
|---|---|---|
| Group | At 0 h | At 24 h |
| IC | Paclitaxel (0.03 μM) and 24 h | CM and 72 h incubation |
| | incubation | |
| | Paclitaxel (0.1 μM) and 24 h | |
| | incubation | |
| | Paclitaxel (0.3 μM) and 24 h | |
| | incubation | |
| | Paclitaxel (1.0 μM) and 24 h | |
| | incubation | |
| | Paclitaxel (3.0 μM) and 24 h | |
| | incubation | |
| IIC | CM and 24 h incubation | Compound A (IC₅₀ = 1 μM) and |
| | | 72 h incubation |
| IIIC | CM and 24 h incubation | Sunitinib (Sutent ®, |
| | | IC₅₀ = 7.8 μM) and 72 h |
| | | incubation |
| IVC | Paclitaxel (0.03 μM) and 24 h | Compound A (IC₅₀ = 1 μM) |
| | incubation | and 72 h incubation |
| | Paclitaxel (0.1 μM) and 24 h | |
| | incubation | |
| | Paclitaxel (0.3 μM) and 24 h | |
| | incubation | |
| | Paclitaxel (1.0 μM) and 24 h | |
| | incubation | |
| | Paclitaxel (3.0 μM) and 24 h | |
| | incubation | |
| VC | Paclitaxel (0.03 μM) and 24 h | Sunitinib (Sutent ®, |
| | incubation | IC₅₀ = 7.8 μM) and 72 h |
| | Paclitaxel (0.1 μM) and 24 h | incubation |
| | incubation | |
| | Paclitaxel (0.3 μM) and 24 h | |
| | incubation | |
| | Paclitaxel (1.0 μM) and 24 h | |
| | incubation | |
| | Paclitaxel (3.0 μM) and 24 h | |
| | incubation | |
| VIC | Vehicle control and 24 h | CM and 72 h incubation |
| | incubation | |

The combination index as evaluated for the combination groups is shown in Table 11. The results are shown in FIG. 14.

TABLE 11
CI values for combination groups in MDA-MB-468

| Group | CI |
|---|---|
| IVC | 0.3-0.8 |
| VC | 0.5-1.0 |

The combination of Paclitaxel and Compound A was comparatively more synergistic than Paclitaxel and Sutent as indicated by the combination index in the TNBC cell line MDA-MB-468.

The invention has been described It should be noted that, as used in this specification and the appended claims, the singular forms “a”, “an”, and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

All publications and patent applications in this specification are indicative of the level of ordinary skill in the art to which this invention pertains.

The invention has been described with reference to various specific and preferred embodiments and techniques. However, it should be understood that many variations and modifications may be made while remaining within the spirit and scope of the invention.

1-23. (canceled)

24. A method of treating triple negative breast cancer in a subject comprising administering to the subject a therapeutically effective amount of paclitaxel or its pharmaceutically acceptable salt and a therapeutically effective amount of the CDK inhibitor selected from the compounds of formula I or a pharmaceutically acceptable salt thereof; 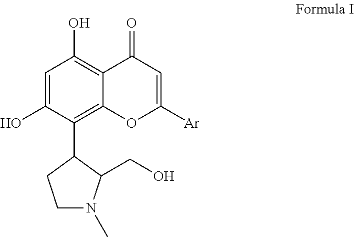
wherein Ar is phenyl, which is unsubstituted or substituted by 1, 2, or 3 identical or different substituents selected from: halogen selected from chlorine, bromine, fluorine or iodine; nitro, cyano, C₁-C₄-alkyl, trifluoromethyl, hydroxyl, C₁-C₄-alkoxy, carboxy, C₁-C₄-alkoxycarbonyl, CONH₂ or NR₁R₂; wherein R₁ and R₂ are each independently selected from hydrogen or C₁-C₄-alkyl.

25. The method according to claim 24, wherein the CDK inhibitor is a compound of formula I or a pharmaceutically acceptable salt thereof; wherein the phenyl group is substituted by 1, 2, or 3 identical or different substituents selected from: halogen selected from chlorine, bromine, fluorine or iodine; C₁-C₄-alkyl or trifluoromethyl.

26. The method according to claim 25, wherein the CDK inhibitor is a compound of formula I or a pharmaceutically acceptable salt thereof; wherein the phenyl group is substituted by 1, 2, or 3 halogens selected from chlorine, bromine, fluorine or iodine.

27. The method according to claim 26, wherein the CDK inhibitor is a compound of formula I or a pharmaceutically acceptable salt thereof; wherein the phenyl group is substituted by chlorine.

28. The method according to claim 27, wherein the CDK inhibitor represented by compound of formula I is (+)-trans-2-(2-Chloro-phenyl)-5,7-dihydroxy-8-(2-hydroxymethyl-1-methyl-pyrrolidin-3-yl)-chromen-4-one hydrochloride (Compound A).

29. The method according to claim 25, wherein the CDK inhibitor is a compound of formula I or a pharmaceutically acceptable salt thereof; wherein the phenyl group is disubstituted with a chloro and a trifluoromethyl group.

30. The method according to claim 29, wherein the CDK inhibitor represented by compound of formula I is (+)-trans-2-(2-Chloro-4-trifluoromethylphenyl)-5,7-dihydroxy-8-(2-hydroxymethyl-1-methyl-pyrrolidin-3-yl)-chromen-4-one hydrochloride (compound B).

31. The method according to claim 24, wherein a therapeutically effective amount of paclitaxel, or its pharmaceutically acceptable salt; and a therapeutically effective amount of the CDK inhibitor represented by a compound of formula I or a pharmaceutically acceptable salt thereof; are administered sequentially to the subject in need thereof.

32. The method according to claim 31, wherein paclitaxel, or its pharmaceutically acceptable salt is administered prior to the CDK inhibitor represented by a compound of formula I or a pharmaceutically acceptable salt thereof.

33. The method according to claim 24, wherein therapeutic synergy is exhibited on administration of paclitaxel and the CDK inhibitor.
